(19) United States
(12) Patent Application Publication
(10) Pub. No.: US 2020/0270240 A1
(43) Pub. Date: Aug. 27, 2020

(54) BENZOTHIAZOLE AND PYRIDOTHIAZOLE COMPOUNDS AS SUMO ACTIVATORS

(71) Applicant: ICAHN SCHOOL OF MEDICINE AT MOUNT SINAI, New York, NY (US)

(72) Inventors: Roger Hajjar, New York, NY (US); Robert Devita, New York, NY (US)

(21) Appl. No.: 16/629,672

(22) PCT Filed: Jul. 20, 2018
(86) PCT No.: PCT/US18/43008

§ 371 Date: Jan. 9, 2020

Related U.S. Application Data
(60) Provisional application No. 62/535,691, filed on Jul. 21, 2017.

(51) Int. Cl. C07D 417/12; C07D 513/04

(52) U.S. Cl. CPC C07D 417/12; C07D 513/04

(57) ABSTRACT

Provided are SUMO activators, which can enhance SUMOylation of SERCA2a, which are useful in the treatment of heart failure, cardiovascular diseases, cancer, neurodegenerative disorders, viral infection, bacterial infection, liver disease, inflammation, and other diseases.

### TECHNICAL FIELD

The present application relates to SUMO activators, which can enhance SUMOylation of SERCA2a, which are useful in the treatment of heart failure, cardiovascular diseases, cancer, neurodegenerative disorders, viral infection, bacterial infection, liver disease, inflammation, and other diseases.

### BACKGROUND

Heart failure (HF) remains a leading cause of death in Western countries and the development of new therapeutic agents for HF has been challenged. The recent major advances in the understanding of molecular signaling in the cardiac myocytes under the pathological stress has suggested the way for different approaches to treating heart disease, in particular to regulate intrinsic targets on the intracellular side. The calcium-transporting ATPase ATP2A2 (SERCA2a) is ATPase responsible for Ca²⁺ re-uptake during excitation-contraction coupling. A characteristic of heart failure is impaired Ca²⁺ uptake resulting from decreased expression and reduced activity of SERCA2a. To this end, restoration of SERCA2a expression by gene transfer can be effective in improving cardiac function in animal models and heart-failure patients. It was found that the levels and activity of SERCA2a in cardiac myocytes are modulated by small ubiquitin-like modifier type 1 (SUMO I)-mediated unique post-translational modification (PTM), named SUMOylation (Kho C, Lee A, Jeong D, Oh J C; Chaanine A H, Kizana E, Park W J, Hajjar R J, “SUMO1-dependent modulation of SERCA2a in heart failure”, Nature 2011 Sep. 7; 477(7366):601-5). SERCA2a is SUMOYLated by SUMO1 at two specific sites Lysine 480 and 585. The levels of SUMO1 and the SUMOylation of SERCA2a itself were greatly reduced in failing hearts. SUMO1 restitution by adeno-associated-virus-mediated gene delivery maintained the protein abundance of SERCA2a and markedly improved cardiac function in mice with heart failure. This effect was comparable to SERCA2A gene delivery. Since it has been shown that SUMO1 enhances the stability and the ATPase activity of SERCA2a, its decrease causes further dysfunction of SERCA2a and further worsening of dysfunction. Further, gain of function experiments by transgenesis and gene therapy showed that SUMO1 gene therapy rescues contractile function and improves mortality in models of heart failure. To this end, there is a need to develop new small molecules that increase SERCA2a SUMOylation, which are useful for treating HF.

Further, induction of SUMOylation has also been implicated in the treatment of cancer (Kira Bettermann, Martin Benesch, Serge Weis, Johannes Haybaeck. SUMOylation in carcinogenesis. Cancer Letters (2012) 316, 113-125), neurodegenerative disorders such as Huntington's disease (Steffan, J. S. et al. SUMO modification of Huntingtin and Huntington's disease pathology. Science (2004) 304, 100-104), Parkinson's disease (Dorval, V., Fraser, P. E. Small ubiquitin-like modifier (SUMO) modification of natively unfolded proteins tau and a-synuclein. J. Biol. Chem. (2006) 281, 9919-9924), Alzheimer's disease (Zhang, Y. Q. and Sarge, K. D. Sumoylation of amyloid precursor protein negatively regulates Ab aggregate levels. (2008) Biochem. Biophys. Res. Commun. 374, 673-678), and amyotrophic lateral sclerosis (ALS) (Fei, E. et al. SUMO-1 modification increases human SOD1 stability and aggregation. Biochem. Biophys. Res. Commun. (2006) 347, 406-412), viral and bacterial infection (Bekes M, Drag M. Trojan horse strategies used by pathogens to influence the small ubiquitin-like modifier (SUMO) system of host eukaryotic cells. J Innate Immun. (2012) 4, 159-67), liver disease (Guo W H, Yuan L H, Xiao Z H, Liu D, Zhang J X. Overexpression of SUMO-1 in hepatocellular carcinoma: a latent target for diagnosis and therapy of hepatoma. J Cancer Res Clin Oncol. (2011) 137, 533-41), and inflammation (Pascual G, Fong A L, Ogawa S, Gamliel A, Li A C, Perissi V, Rose D W, Willson T M, Rosenfeld M Q Glass C K. A SUMOylation-dependent pathway mediates transrepression of inflammatory response genes by PPAR-gamma. Nature (2005) 437, 759-63).

To this end, there is a need to develop new small molecules that increase SERCA2a SUMOylation, which are useful for treating HF. This application addresses this need and others.

### SUMMARY

The present application provides compounds described herein, or a pharmaceutically acceptable salt thereof, which are useful as SUMO activators.

The present application further provides a method of treating heart failure, cardiac hypertrophy, myocarditis, myocardial infarction, ischemia, cardiac arrhythmias, vascular rhexis, cardiac arrhythmia, valvulopathy, diastolic dysfunction, hypertension, cancer, neurodegenerative disorders, viral infection, bacterial infection, liver disease, or inflammation in a patient in need thereof, comprising administering to said patient a therapeutically effective amount of a compound described herein, or a pharmaceutically acceptable salt thereof.

The present application also provides a method which further comprises administering to the patient an adeno-associated vector (AAV) comprising SERCA2a.

The present application further provides a method of activating SUMO1, comprising contacting comprising contacting a cell with a compound, salt, or composition described herein, in an amount effective to activate SUMO1.

The present application further provides a compound or salt as described herein for use in any of the methods described herein.

The present application further provides use of a compound or salt as described herein for manufacture of a medicament for use in any of the method described herein.

The present application further provides a pharmaceutical composition comprising any of the compounds described herein, or a pharmaceutically acceptable salt thereof, and at least one pharmaceutically acceptable carrier.

### DETAILED DESCRIPTION

The present application provides compounds described herein, or a pharmaceutically acceptable salt thereof, which are useful as SUMO activators. The present application further provides a method of treating heart failure, cardiac hypertrophy, myocarditis, myocardial infarction, ischemia, cardiac arrhythmias, vascular rhexis, cardiac arrhythmia, valvulopathy, diastolic dysfunction, hypertension, cancer, neurodegenerative disorders, viral infection, bacterial infection, liver disease, or inflammation in a patient in need thereof, comprising administering to said patient a therapeutically effective amount of a compound described herein, or a pharmaceutically acceptable salt thereof.

In some embodiments, the heart failure is selected from congestive heart failure (CHF), chronic heart failure, and ischemic heart failure.

Cancers include, but are not limited to, solid tumors such as breast, ovarian, prostate, lung, kidney, gastric, colon, testicular, head and neck, pancreas, brain, melanoma, and other tumors of tissue organs and cancers of the blood cells, such as lymphomas and leukemias, including acute myelogenous leukemia, chronic lymphocytic leukemia, T cell lymphocytic leukemia, and B cell lymphomas.

Inflammatory disorders include, but are not limited to, transplant rejection, including skin graft rejection; chronic inflammatory disorders of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung disorders such as asthma, adult respiratory distress syndrome, and chronic obstructive airway disease; inflammatory disorders of the eye including corneal dystrophy, trachoma, onchocerciasis, uveitis, sympathetic ophthalmitis and endophthalmitis; chronic inflammatory disorders of the gums, including gingivitis and periodontitis; tuberculosis; leprosy; inflammatory diseases of the kidney including uremic complications, glomerulonephritis and nephrosis; inflammatory disorders of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune disorders, immune-complex vasculitis, systemic lupus and erythematodes; systemic lupus erythematosus (SLE); and inflammatory diseases of the heart such as cardiomyopathy, ischemic heart disease hypercholesterolemia, atherosclerosis; as well as various other diseases with significant inflammatory components, including preeclampsia; chronic liver failure, brain and spinal cord trauma, and cancer. There may also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent used in cancer chemotherapy.

Neurodegenerative disorders include, but are not limited to Huntington's disease, Parkinson's disease, Alzheimer's disease, and amyotrophic lateral sclerosis (ALS).

Viral infections, include but are not limited to, infections by a hepatitis virus (e.g., hepatitis B or C), human immunodeficiency virus (HIV), rhinovirus, herpes-zoster virus (VZV), herpes simplex virus (e.g., HSV-1 or HSV-2), cytomegalovirus (CMV), vaccinia virus, influenza virus, encephalitis virus, hantavirus, arbovirus, West Nile virus, human papilloma virus (HPV), Epstein-Bar virus, and respiratory syncytial virus.

Liver diseases include, but are not limited to liver cirrhosis, alcoholic liver cirrhosis, fatty liver, toxipathic liver diseases, and acute and chronic hepatitis.

The compounds described herein can activate SUMO1. Accordingly, the present application further provides a method of activating SUMO1, comprising contacting comprising contacting a cell with a compound, salt, or composition described herein, in an amount effective to activate SUMO1. The contacting can be done in vivo or in vitro. In further embodiments, the compounds of the present application can be used to activate SUMO1 in an individual in need of the activation by administering a compound, salt, or composition described herein, in an amount effective to activate SUMO1.

The present application further provides a pharmaceutical composition comprising any of the compounds described herein, or a pharmaceutically acceptable salt thereof, and at least one pharmaceutically acceptable carrier.

The present application further provides any of the compounds described herein, or a pharmaceutically acceptable salt thereof.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Compounds of the present application also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone enol pairs, amide—imidic acid pairs, lactam lactim pairs, enamine imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present application can also include all isotopes of atoms occurring in the intermediates or final compounds. Isotopes include those atoms having the same atomic number but different mass numbers. For example, isotopes of hydrogen include tritium and deuterium.

The term, “compound,” as used herein is meant to include all stereoisomers, geometric iosomers, tautomers, and isotopes of the structures depicted. Compounds herein identified by name or structure as one particular tautomeric form are intended to include other tautomeric forms unless otherwise specified.

All compounds, and pharmaceutically acceptable salts thereof, can be found together with other substances such as water and solvents (e.g. hydrates and solvates) or can be isolated.

In some embodiments, the compounds of the present application, or salts thereof, are substantially isolated. By “substantially isolated” is meant that the compound is at least partially or substantially separated from the environment in which it was formed or detected. Partial separation can include, for example, a composition enriched in the compounds of the present application. Substantial separation can include compositions containing at least about 50%, at least about 60%, at least about 70%, at least about 80%, at least about 90%, at least about 95%, at least about 97%, or at least about 99% by weight of the compounds of the present application, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Combination Therapy

In some embodiments, a compound of the present application, or a pharmaceutically acceptable salt thereof, can be used in combination with another therapeutic agent to treat diseases such as cancer and/or neurological disorders. For example, the additional agent can be a therapeutic agent that is art-recognized as being useful to treat the disease or condition being treated by the compound of the present application. The additional agent also can be an agent that imparts a beneficial attribute to the therapeutic composition (e.g., an agent that affects the viscosity of the composition).

The combination therapy contemplated by the invention includes, for example, administration of a compound of the present application, or a pharmaceutically acceptable salt thereof, and additional agent(s) in a single pharmaceutical formulation as well as administration of a compound of the present application, or a pharmaceutically acceptable salt thereof, and additional agent(s) in separate pharmaceutical formulations. In other words, co-administration shall mean the administration of at least two agents to a subject so as to provide the beneficial effects of the combination of both agents. For example, the agents may be administered simultaneously or sequentially over a period of time.

The additional therapeutic agent can be any therapeutic agent useful for the treatment of the disease states of the methods described herein. The additional therapeutic agent can be administered simultaneously or sequentially. In another embodiment, the method further comprises administering to the patient a viral expression vector comprising SERCA2a. In some embodiments, the method further comprises administering to the patient an adeno-associated vector (AAV) comprising SERCA2a. For example, vectors useful in the present methods include, but are not limited to those described in US 2011/0256101, which is incorporated herein by reference in its entirety.

In one embodiment, SERCA2 is incorporated into a viral vector to mediate transfer to a cell. Alternatively, a retrovirus, bovine papilloma virus, an adenovirus vector, a lentiviral vector, a vaccinia virus, a polyoma virus, or an infective virus may be used. Similarly, nonviral methods which include, but are not limited to, direct delivery of DNA such as by perfusion, naked DNA transfection, liposome mediated transfection, encapsulation, and receptor-mediated endocytosis may be employed. These techniques are well known to those of skill in the art, and the particulars thereof do not lie at the crux of the present invention and thus need not be exhaustively detailed herein. For example, a viral vector is used for the transduction of pulmonary cells to deliver a therapeutically significant polynucleotide to a cell. The virus may gain access to the interior of the cell by a specific means such as receptor-mediated endocytosis, or by non-specific means such as pinocytosis

The practice of the present application may employ conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

### Pharmaceutical Formulations and Dosage Forms

When employed as pharmaceuticals, the compounds of the present application can be administered in the form of pharmaceutical compositions. These compositions can be prepared in a manner well known in the pharmaceutical art, and can be administered by a variety of routes, depending upon whether local or systemic treatment is desired and upon the area to be treated. Administration routes include, but are not limited, to pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This application also includes pharmaceutical compositions which contain, as the active ingredient, the compound of the present application or a pharmaceutically acceptable salt thereof, in combination with one or more pharmaceutically acceptable carriers (excipients). In some embodiments, the composition is suitable for topical administration. In making the compositions of the present application, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the present application may be milled using known milling procedures such as wet milling to obtain a particle size appropriate for tablet formation and for other formulation types. Finely divided (nanoparticulate) preparations of the compounds of the present application can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the present application can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the present application contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the present application contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the present application contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the present application.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present application. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present application.

The tablets or pills of the present application can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present application can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present application can vary according to, for example, the particular use for which the treatment is made, the manner of administration of the compound, the health and condition of the patient, and the judgment of the prescribing physician. The proportion or concentration of a compound of the present application in a pharmaceutical composition can vary depending upon a number of factors including dosage, chemical characteristics (e.g., hydrophobicity), and the route of administration. For example, the compounds of the present application can be provided in an aqueous physiological buffer solution containing about 0.1 to about 10% w/v of the compound for parenteral administration. Some typical dose ranges are from about 1 □g/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### Synthesis

Compounds of the present application, including salts thereof, can be prepared using known organic synthesis techniques and can be synthesized according to any of numerous possible synthetic routes, for example, by methods analogous to those described in the Examples section.

The reactions for preparing compounds of the present application can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the present application can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC).

Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present application. Cis and trans geometric isomers of the compounds of the present application are described and may be isolated as a mixture of isomers or as separated isomeric forms.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

### Kits

The present application also includes pharmaceutical kits useful, for example, in the treatment or prevention of any of the disease states described herein, which include one or more containers containing a pharmaceutical composition comprising a therapeutically effective amount of a compound of the present application. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### Materials and Methods

### Antibodies and Transfection

The following antibodies are used for immunoblotting and immunoprecipitation (IP): polyclonal anti-SERCA2a (21th century Biochem Inc.), monoclonal anti-GAPDH (Sigma-Aldrich, catalog no. G8795), monoclonal anti-SUMO 1 (Cell Signaling Technology Inc., catalog no. 4930), anti-mouse horseradish peroxidase (HRP) (Pierce Biotechnology Inc., catalog no. 32430), and anti-rabbit HRP (Pierce Biotechnology Inc., catalog no. 32460). YFP-tagged SUMO1, pcDNA3.0-SERCA2a, and HA-tagged Ubc9 plasmids are used for the transfection. The plasmid DNA is amplified in the Escherichia coli strain DH5a and extracted by using a commercial purification kit (Qiagen, catalog no. 12263). Purified plasmid is resuspended in sterile TE buffer (10 mM Tris-HCl and 1 mM EDTA, pH 7.6). Only the preparation highest purity (A260/A280>1.8) is used for transfection. 1 μg of each plasmid is used for transfection. HEK-293 cells (American Type Culture Collection, catalog no. CRL-1873) are grown at 37° C. and under a 5% CO₂ humidified atmosphere in Dulbecco's modified Eagle's medium (DMEM, Cellgro, catalog no. 10-0 13-CM) containing 10% fetal bovine serum (SAFC Bioscience, catalog no. 12107) and 100 i.μ of penicillin/ml and 100 μg of streptomycin/ml. HEK-293 cells are seeded at a density of 3-5×10⁵ cells per 60 mm culture dish in DMEM. The cells are transiently transfected using Lipofectamine 2000 (Invitrogen, catalog no. 11668) with indicated expression plasmids. After 24 hours, the cells are treated with either small molecules or dimethyl sulfoxide (DMSO, Sigma-Aldrich, catalog no. D2650).

### Cardiac Myocyte Isolation

Calcium-tolerant adult rat ventricular myocytes (ARVMs) are obtained from hearts of male Sprague-Dawley rats (250 to 300 g). The heart is excised and perfused with a standard enzymatic technique. ARVMs are plated on multi-chambered plates or culture dishes, pre-coated with laminin 2 μg/cm², at a density of 10⁵ cells/cm² in DMEM without L-glutamine supplemented with 10 mmol/l HEPES, 3.7 mg/ml NaHCO₃, 1 mg/ml glucose, 0.11 mg/ml sodium pyruvate, 2 mg/ml bovine serum albumin, 2 mmol/l L-camitine, 5 mmol/l creatine, 5 mmol/l taurine, 1% penicillin-streptomycin, and 1% gentamycin. Following isolation, cells are allowed to settle for 1 hour. Cultures are incubated at 37° C., in an atmosphere of 5% CO₂-95% air. Fresh medium is added gently as medium is being drawn off until the cultures have been thoroughly washed. Only quiescent, rod-shape cardiac myocytes are selected for IonOptix experiments.

### Example Compounds

Each compound is added at 10 μM. DMSO is used as a control. After 24 hours at 37° C., SERCA2a SUMOylation and functional analysis are determined.

### Cell Image

Cellular permeability and potential activity of the compounds are examined by tracking of the YFP-SUMO1. HEK-293 cells expressing YFP-SUMOI and pcDNA3.0-SERCA2a are incubated with either 10 μM DMSO or 10 μM small molecules for 24 hours at 37° C. Fluorescent signals are monitored by fluorescence microscopy. HA-tagged Ubc9 expressing cells are served as a positive control.

### Cell Shortening/Re-Lengthening

Mechanical properties of ARVMs are assessed using an IonOptix MyoCam® system (IonOptix, Milton, Mass.). In brief, cells are placed in a Warner chamber mounted on the stage of an inverted microscope (Olympus, IX-70) and superfused (1 ml/min at 30° C.) with a buffer containing 131 mM NaCl, 4 mM KCl, 1 mM CaCh, 1 mM MgCh, 10 mM glucose, and 10 mM HEPES, pH 7.4. ARVMs are field stimulated with suprathreshold voltage and at a frequency of 0.5 Hz. The ARVMs being studied is displayed on the computer monitor using an IonOptix MyoCam camera. SoftEdge software (IonOptix) is used to capture changes in cell length during shortening and re-lengthening.

### Intracellular Fluorescence Measurement

ARVMs are placed in a chamber on an Olympus IX-70 inverted microscope and imaged through a Fluor 40x oil objective. ARVMs are exposed to light emitted by a 75 W lamp and passed through either a 360 or a 380 nm filter (bandwidths are ±15 nm), while being stimulated to contract at 0.5 Hz. Fluorescence emissions are detected between 480 and 520 nm by a photomultiplier tube after first illuminating cells at 360 nm for 0.5 s then at 380 nm for the duration of the recording protocol (333 Hz sampling rate). The 360 nm excitation scan is repeated at the end of the protocol and qualitative changes in intracellular Ca²⁺ concentration ((Ca²⁺]ᵢ) are inferred from the ratio of the fluorescence intensity at two wavelengths.

### Immunoblotting

Equal amounts of protein from either small molecule treated or DMSO treated cells or immunoprecipitates are resolved by 7.5% SDS-PAGE and transferred to nitrocellulose membranes (Bio-Rad, catalog no. 162-0112). The membranes are blocked for 1 hour at room temperature with 5% non-fat milk (Cell Signaling Technology Inc., catalog no. 9999) in TBST (10 mM Tris-HCl, 150 mM NaCl, and 0.05% tween-20, pH 8.0). The blots are incubated with specific primary antibodies at 4° C. for overnight. The blots are then washed five times for 10 minutes each with TBST and incubated for 1 hour with HRP-conjugated secondary antibodies in TBST with 5% non-fat milk. After five times TBST washes, the protein bands are visualized with enhanced chemiluminescence (Pierce Biotechnology Inc., catalog no. 32132) and exposed to x-ray film (Denville Scientific Inc., catalog no. E3012). GAPDH expression provided an internal control.

### SERCA2a SUMOylation Assay

Post-transfection (48 hours), the HEK-293 cells are rinsed twice with phosphate-buffered saline (PBS, Cellgro, catalog no. 21-040-CM) and lysed in 1% Nonidet P-40 lysis buffer (Boston Bioproducts, catalog no. BP-119) with 10 mM N-ethylmal eimide (NEM, Sigma-Aldrich, catalog no. N3876) and phosphatase inhibitor cocktail (Complete Mini Tablet, Roche Applied Science, catalog no. 11836153001). 2 mg of protein are mixed with the anti-SERCA2a antibodies for overnight at 4° C. in lysis buffer. Pre-washed protein A-Separose beads (Pierce Biotechnology Inc., catalog no. 20333) is added to each sample and incubated 1 hour at 4° C. with gentle rocking. Immunocomplexes are washed with lysis buffer three times and precipitated by centrifugation at 12000×g for 10 seconds. The immunocomplexes are resuspended in SDS sample buffer and subjected to immunoblotting. Controls for the immunoprecipitations are performed using an anti-rabbit IgG equal to that of the primary precipitating antibody. Ten percent of whole cell lysates used in the immunoprecipitation is loaded for subsequent immunoblotting.

### Statistics

Data are obtained from experiments performed two or three times and values are presented as mean±standard deviation (SD). The p value was calculated by analysis of variance, followed by Student's t test. Difference between the groups of data are considered statistically significant when p<0.05.

### EXAMPLES

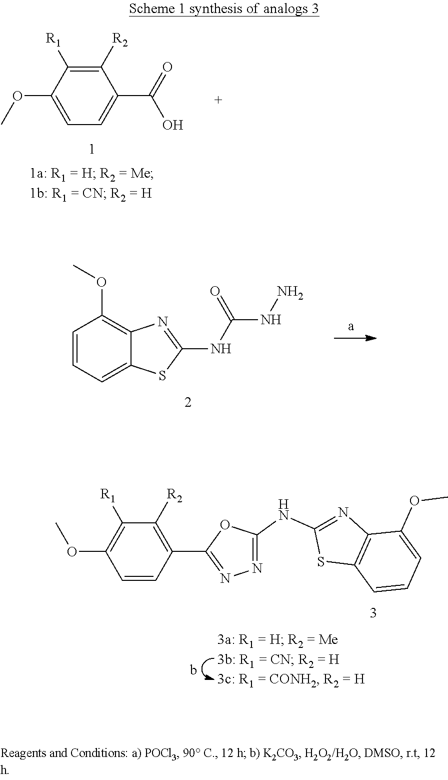

The synthesis of N106 analogs 3a-c are shown in Scheme 1, the 1,3,4-oxadiazole ring was constructed by cyclodehydration of N, N-diacyl-hydrazines between benzoic acid 1a,b and hydrazinecarboxamide 2 in POCl₃.^{[1]} Analogs 3c was obtained by hydrolysis of 3b in the presence of H₂O₂/H₂O/K₂CO₃.

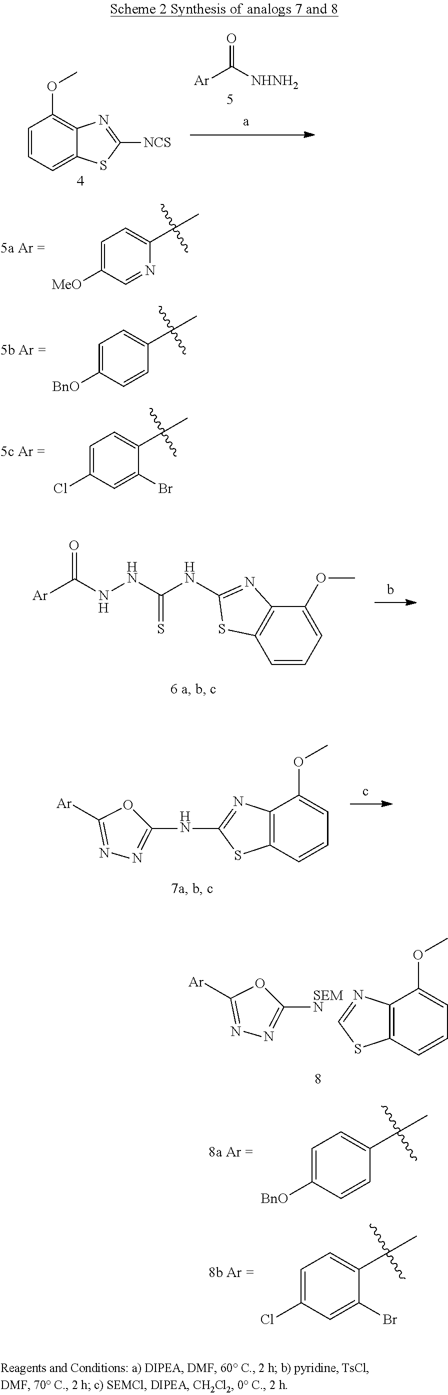

The synthesis of analogs 7 and 8 are outlined in Scheme 2. The N, N-diacyl-hydrazine 6a-c was synthesized via the addition between isothiocyanato 4^{[2]} and hydrazinecarboxamide 5a-c^{[3]}. Intramolecular cyclization of 6a-c in the presence of TsCl/pyridine gave the desired 1,3,4-oxadiazole 7a-c^{[4]}, then SEM protection of amine 7b-c by SEMC1 to give compound 8a-b.

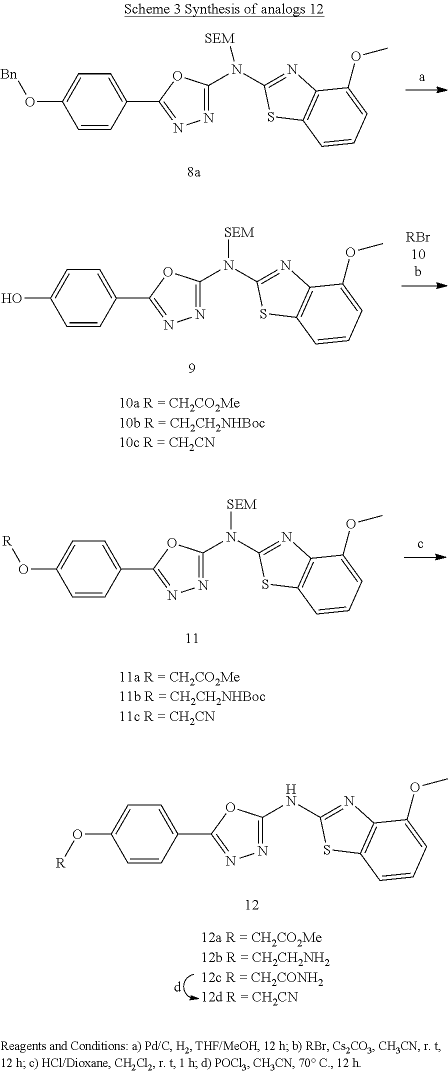

The synthesis of analogs 12a-d are demonstrated in Scheme 3. Hydrogenation of 8a to remove the benzyl group catalyzed by Pd/C under H2 atmosphere give the phenol 9 ^{[5]}, Then alkylation with bromoalkane 10 a-c to yield compound 11 a-c, followed by remove of the protecting group SEM in HCl/dioxane^{[6]} to generate the corresponding compound 12a-c. The dehydration of amide 12c in POCl₃ give the nitrile 12d.

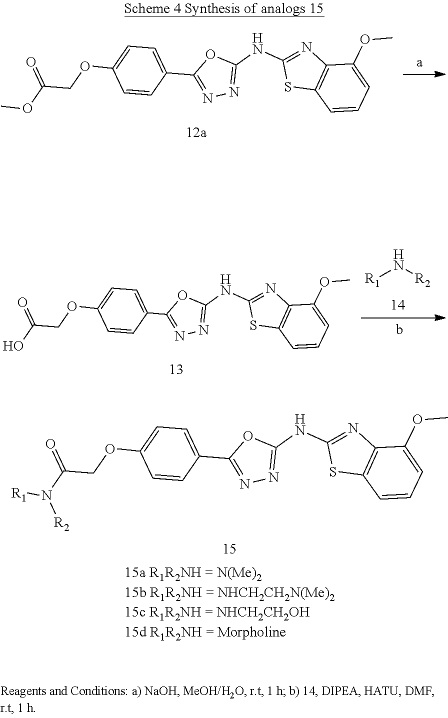

A series of amide analogs 15a-d was synthesized as shown in Scheme 4. Hydrolysis of methyl ester 12a by NaOH/H₂O/MeOH give the corresponding acid 13, which was then coupled with varies of amines in the presence of HATU/DIPEA to yield the amide 15a-d.

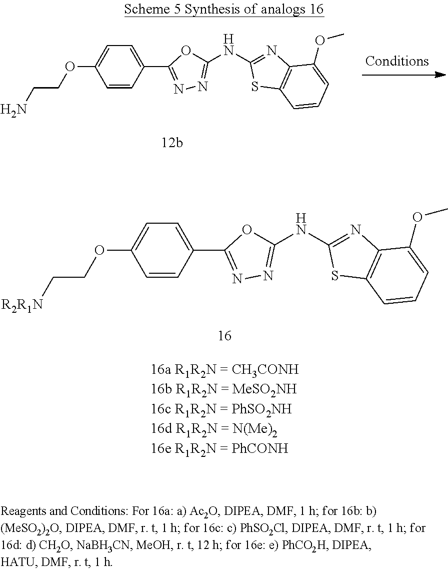

The synthesis of analogs 16a-e is outlined in Scheme 5. Acylation of 12b by acetic anhydride give the acetate 16a, mesylation of 12b by methanesulfonic anhydride in DMF yield 16b, sulfonylation of 12b with phenyl sulfonyl chloride give compound 16c; then a cascade reductive amination between 12b and aldehyde result the formation of 16d, finally amidation of 12b by couple with benzoic acid in the presence of HATU/DIPEA to give product 16e.

TBO1B Analogs:

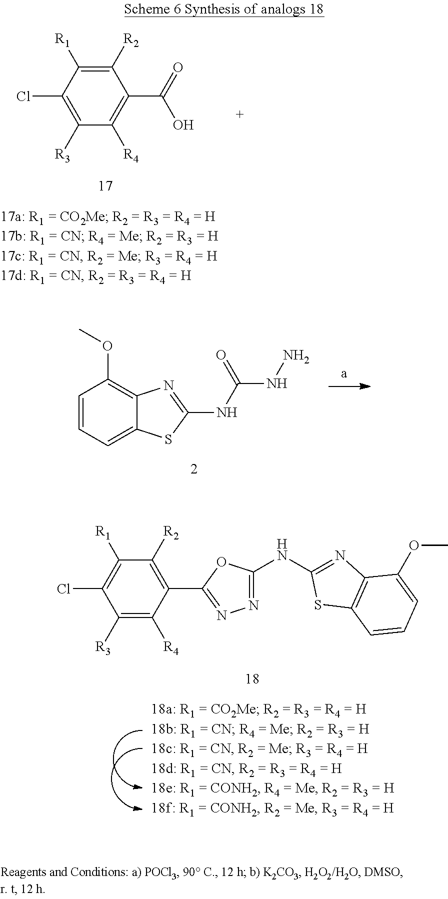

A series of analogs 18a-f was synthesized as listed in Scheme 6. According to previous method for 3a-c, 18a-d was synthesized by cyclodehydration of formed N, N-diacyl-hydrazines intermediate between benzoic acid 17a-d and hydrazinecarboxamide 2 in POCl₃. 18b-c was then subject to hydrolysis by H₂O₂/K₂CO₃/H₂O to generate the amide 18e-f.^{[7]}

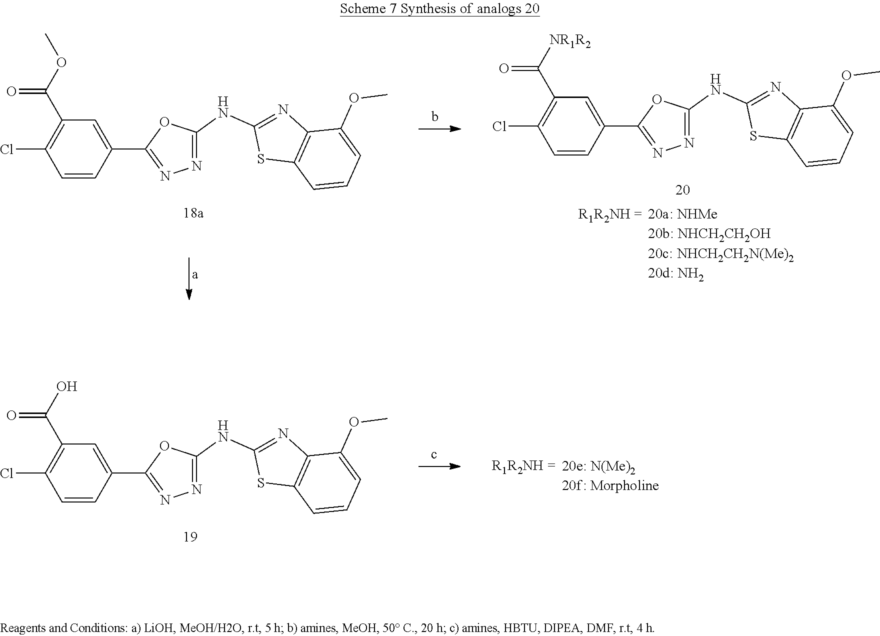

The synthesis of analogs 20a-f was shown in Scheme 7. 20a-d was synthesized via direct ammonolysis of 18a with corresponding amines. The hydrolysis of 18a with LiOH/MeOH to give aryl acid 19, which was coupling with corresponding amines in the presence of HBTU/DIPEA to give amide 20e-f.

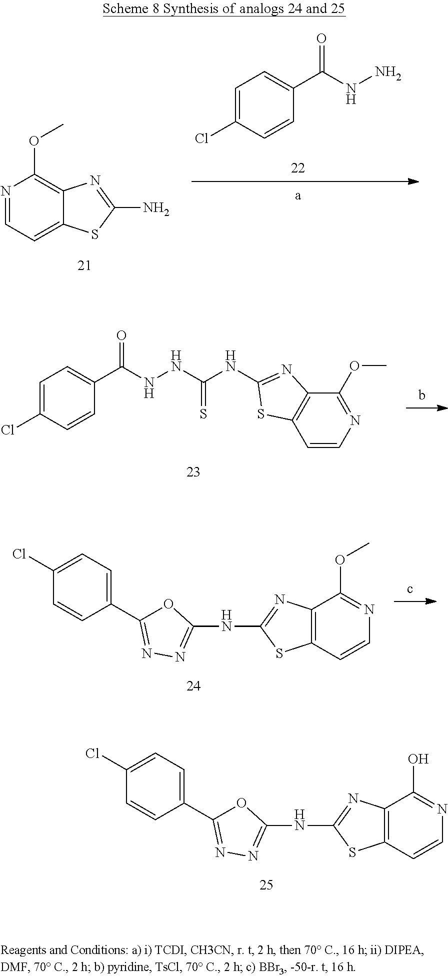 [SMILES: COc1nccc2sc(N)nc12 ; NNC(=O)c1ccc(Cl)cc1 ; COc1nccc2sc(NC(=S)NNC(=O)c3ccc(Cl)cc3)nc12 ; COc1nccc2sc(Nc3nnc(-c4ccc(Cl)cc4)o3)nc12 ; Oc1nccc2sc(Nc3nnc(-c4ccc(Cl)cc4)o3)nc12]

Scheme 8 shown the synthesis of analogs 24 and 25. Similar to previous synthesis of 7a-c, compound 24 was synthesized via a three steps reaction: the primary amine 21 was converted into isothiocyanato by TCDI, followed by addition with hydrazine 22 to give the corresponding hydrazinecarbothioamide 23, then intramolecular condensation/cyclization in the presence of TsCl/Pyridine to yield the desired product 24. Then demethylation of 24 by using BBr₃ to give analog 25.^{[8]}

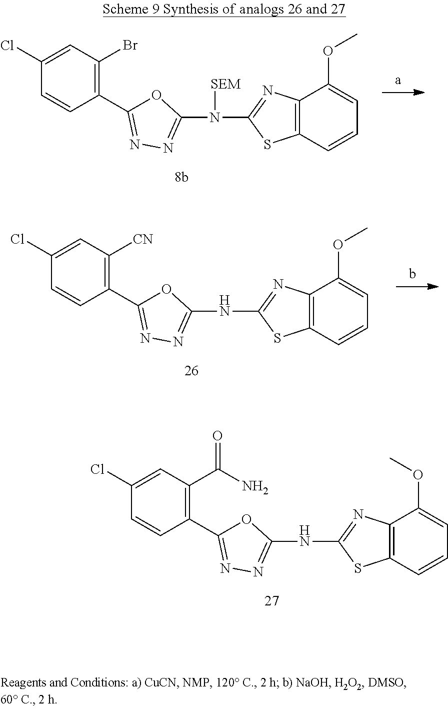 [SMILES: COc1cccc2sc(N(COCC[Si](C)(C)C)c3nnc(-c4ccc(Cl)cc4Br)o3)nc12 ; COc1cccc2sc(Nc3nnc(-c4ccc(Cl)cc4C#N)o3)nc12 ; COc1cccc2sc(Nc3nnc(-c4ccc(Cl)cc4C(N)=O)o3)nc12]

The synthesis of analogs 26 and 27 are shown in Scheme 9. The bromoaryl 8b was converted into nitrile 26 by using CuCN in NMP under high temperature with the loss of protecting group during the reaction. Compound 26 was then subject to hydrolysis by H₂O₂ to give the amide 27.

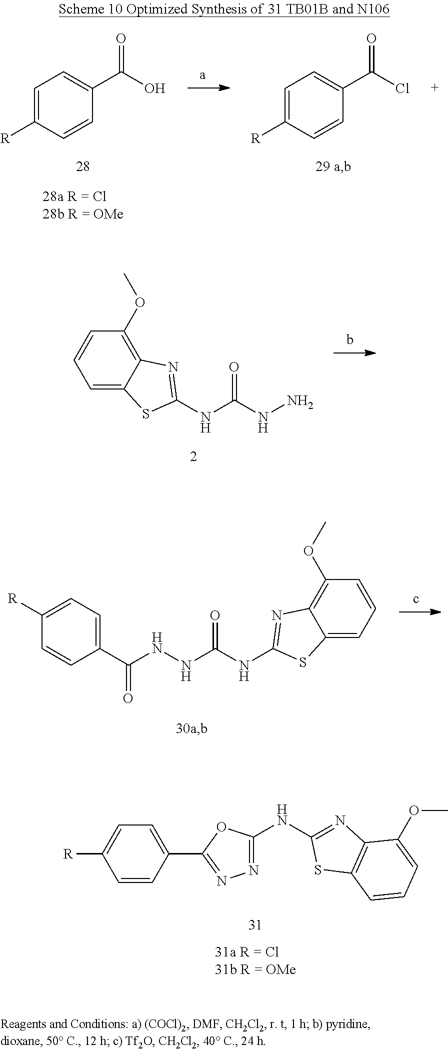

A improved route of the synthesis of two active analogs 31a-b was developed as shown in Scheme 10. The benzoic acid 28a-b was converted into acyl chloride 29a-b by using oxalyl chloride catalyzed by DMF, then amidation by hydrazinecarboxamide 2 to give the compound 30a-b. The intramolecular cyclodehydration of N, N′-diacyl-hydrazines 30a-b in the presence of trifluoromethylsulfonic anhydride in dichloromethane give the desired product 31a-b in 90% yield.^{[9]}

### EXPERIMENTAL SECTION

Reactions in anhydrous solvents were carried out in glassware that was flame-dried or oven-dried. Unless noted, reactions were magnetically stirred and conducted under an atmosphere. Air-sensitive reagents and solutions were transferred via syringe and were introduced to the reaction vessel through rubber septa. Solids were introduced under a positive pressure of Ar. Temperatures, other than room temperature (rt); refer to bath temperatures unless otherwise indicated. All commercially obtained solvents and reagents were used as received. Deionized water was used for all aqueous reactions, work-ups, and for the preparation of all aqueous solutions. The phrase “concentrated in vacuo” refers to removal of solvents by means of a Buchi rotary-evaporator attached to a variable vacuum pump followed by pumping to a constant weight (<1 Torr). Proton and carbon nuclear magnetic resonance (NMR) spectra were obtained on a Bruker Avance 600 (600 MHz). Chemical shifts are reported in ppm (δ). ¹H NMR data are reported as follows: chemical shift (multiplicity, coupling constant (Hz), number of hydrogens). Multiplicities are denoted accordingly: s (singlet), d (doublet), dd (doublet of doublets), ddd (doublet of doublet of doublets), dt (doublet of triplets), tt (triplet of triplets), dq (doublet of quartets), t (triplet), q (quartet), p (pentet), m (multiplet). High resolution mass spectra (LCMS) were obtained using an Agilent 1200 Series Rapid Resolution LC/MS. The chromatography was performed by using Teledyne ISCO RediSep normal phase (40-60 microns) silica Gel disposable flash columns using a Teledyne ISCO Combiflash Rf purification system. Preparative reversed phase chromatography was carried out by using a Gilson 271 liquid handler coupled to a UV-vis159 Gilson detector, Gilson 322 pump and with a Luna 10 μm C18(2) 100A AXIA Packed column 100×21.2 mm. Mobile phase: linear gradient from 5%-90% CH₃CN in H₂O (0.1% formic acid), at flow rate of 20 mL/min.

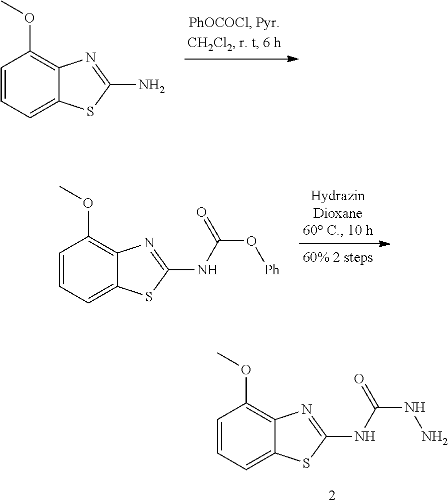 [SMILES: COc1cccc2sc(N)nc12 ; COc1cccc2sc(NC(=O)Oc3ccccc3)nc12 ; COc1cccc2sc(NC(=O)NN)nc12]

### N-(4-methoxybenzo[d]thiazol-2-yl)hydrazinecarboxamide (2)

To a solution of amine 4-methoxybenzo[d]thiazol-2-amine (1 g, 5.55 mmol, 1 equiv.) and pyridine (0.8 mL, 1.5 equiv.) in CH₂Cl₂ (20 mL) was added with phenyl chloroformate (0.96 g, 1.1 equiv.) slowly at 0° C., the mixture was stirred at room temperature for 6 hour, then concentrated and purified via FCC (Hexanes:EtOAc, 2:1) to give product phenyl (4-methoxybenzo[d]thiazol-2-yl)carbamate. ¹H-NMR (600 MHz, CDCl₃): δ 7.44-7.41 (m, 3H), 7.30-7.26 (m, 2H), 7.24-7.22 (m, 2H), 6.91-6.89 (d, 1H), 3.99 (s, 3H). LCMS (TOF-ESI) for C₁₅H₁₂N₂O₃S, Calculated: 301.0639, Found [M+H]⁺ for 301.0644. The above obtained product phenyl (4-methoxybenzo[d]thiazol-2-yl)carbamate in Dioxane (20 mL) was added with hydrazine (5 mL, 5 equiv.) and the mixture was stirred at 60° C. for 10 h. After cooled to room temperature, the suspension mixture was filtered and the cake was washed with CH₂Cl₂ to give product N-(4-methoxybenzo[d]thiazol-2-yl)hydrazinecarboxamide 2 as white solid (0.78 g, 60% over 2 steps). ¹H-NMR (600 MHz, d6-DMSO): δ 7.43-7.42 (d, 1H), 7.15 (s, 1H), 6.93-6.92 (d, 1H), 3.88 (s, 3H). LCMS (TOF-ESI) for C₉H₁₀N₄O₂S, Calculated for [M+H]: 239.0596; Found [M+H]⁺ for 239.0598.

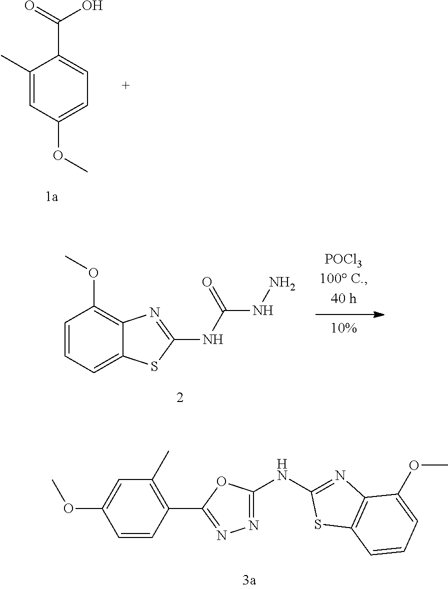

### 5-(4-methoxy-2-methylphenyl)-N-(4-methoxybenzo[d]thiazol-2-yl)-1,3,4-oxadiazol-2-amine (3a)

A solution of 4-methoxy-2-methylbenzoic acid 1a (0.3 g, 1.81 mmol, 1 equiv.) in POCl₃ (10 mL) was stirred at 90° C. under Ar for 1 h, then cooled to room temperature and N-(4-methoxybenzo[d]thiazol-2-yl)hydrazinecarboxamide 2 (0.43 g, 1 equiv.) was added. The mixture was stirred for 40 h at 90° C. under Ar. After cooled to room temperature, the reaction mixture was concentrated under high vacuum. The residue was treated with Et₃N and then concentrated and washed with water and subjected to FCC (Hexanes/EtOAc, 2:1) to give product as light pink solid 5-(4-methoxy-2-methylphenyl)-N-(4-methoxybenzo[d]thiazol-2-yl)-1,3,4-oxadiazol-2-amine 3a (66 mg, 10%). ¹H-NMR (600 MHz, CDCl₃): δ 7.92-7.91 (d, 1H), 7.22-7.19 (t, 1H), 7.16-7.15 (d, 1H), 6.89-6.88 (d, 1H), 6.86-6.84 (m, 2H), 3.96 (s, 3H), 3.87 (s, 3H), 2.75 (s, 3H); ¹³C NMR (125 MHz, CDCl₃): δ 164.1, 160.7, 160.1, 145.6, 139.6, 129.7, 124.2, 116.5, 115.6, 113.5, 111.0, 107.6, 55.3, 54.8, 22.1; LCMS (TOF-ESI) for C₁₅H₁₆N₄O₃S, Calculated for [M+H]: 369.1016; Found [M+H]⁺ for 369.1021.

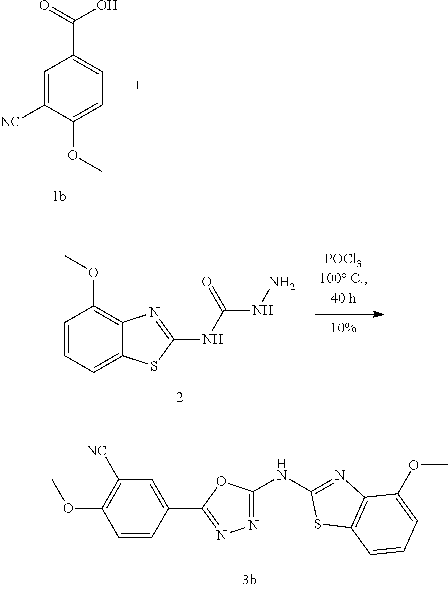

### 2-methoxy-5-(5-((4-methoxybenzo[d]thiazol-2-yl)amino)-1,3,4-oxadiazol-2-yl)benzonitrile (3b)

A solution of 3-cyano-4-methoxybenzoic acid 1b (0.3 g, 1.694 mmol, 1 equiv.) in POCl₃ (10 mL) was stirred at 90° C. under Ar for 1 h, then cooled to room temperature and N-(4-methoxybenzo[d]thiazol-2-yl)hydrazinecarboxamide 2 (0.41 g, 1 equiv.) was added. The mixture was stirred for 40 h at 90° C. under Ar. After cooled to room temperature, the reaction mixture was concentrated under high vacuum. The residue was treated with Et₃N and then concentrated and washed with water and subjected to FCC (CH₂Cl₂/MeOH, 20:1) to give product as light grey solid 2-methoxy-5-(5-((4-methoxybenzo[d]thiazol-2-yl)amino)-1,3,4-oxadiazol-2-yl)benzonitrile 3b (50 mg, 10%). ¹H-NMR (600 MHz, d₆-DMSO): δ 8.20 (s, 1H), 8.15-8.14 (d, 1H), 7.47-7.45 (d, 1H), 7.36-7.34 (d, 1H), 7.20-7.17 (t, 1H), 6.88-6.87 (d, 1H), 3.96 (s, 3H), 3.41 (s, 3H); ¹³C NMR (125 MHz, d₆-DMSO): δ 173.0, 163.4, 162.7, 156.3, 147.2, 136.7, 132.7, 131.4, 128.3, 124.3, 116.2, 115.2, 114.3, 113.2, 107.9, 101.4, 56.9, 55.2; LCMS (TOF-ESI) for C₁₈H₁₃N₅O₃S, Calculated for [M+H]: 380.0812; Found [M+H]⁺ for 380.0821.

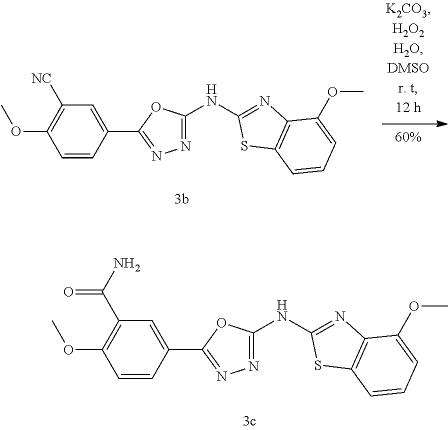

### 2-methoxy-5-(5-((4-methoxybenzo[d]thiazol-2-yl)amino)-1,3,4-oxadiazol-2-yl)benzamide (11)

A solution of 2-methoxy-5-(5-((4-methoxybenzo[d]thiazol-2-yl)amino)-1,3,4-oxadiazol-2-yl)benzonitrile 3b (10 mg, 0.0264 mmol, 1 equiv.) and K₂CO₃ (8 mg, 2 equiv.) in DMSO (1 mL) was added with H₂O₂ (8M in water, 0.02 mL, 5 equiv.), the mixture was stirred at room temperature for 12 h till LC/MS shown the completion of conversion. After completion of reaction, the mixture was filtered and concentrated under high vacuum, the residue was washed with water and dried under high vacuum to provide product 2-methoxy-5-(5-((4-methoxybenzo[d]thiazol-2-yl)amino)-1,3,4-oxadiazol-2-yl)benzamide 3c as light yellow solid (7 mg, 60%). ¹H-NMR (600 MHz, d₆-DMSO): δ 8.32 (s, 1H), 8.03-8.01 (d, 1H), 7.76 (s, 1H), 7.72 (s, 1H), 7.42-7.41 (d, 1H), 7.34-7.33 (d, 1H), 7.20 (t, 1H), 7.07-7.06 (d, 1H), 3.97 (s, 3H), 3.94 (s, 3H); LCMS (TOF-ESI) for C₁₈H₁₅N₅O₄S, Calculated for [M+H]: 398.0918; Found [M+H]⁺ for 398.0928.

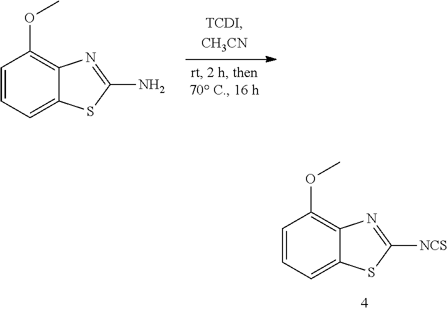

### 2-isothiocyanato-4-methoxybenzo[d]thiazole (4)

To a solution of 4-methoxybenzo[d]thiazol-2-amine (10.8 g, 60.0 mmol) in CH₃CN (200 mL) was added TCDI (11.2 g, 63.0 mmol) in portions. After addition, the mixture was stirred for 2 h at room temperature, then heated to 70° C. and stirred overnight under N₂. After cooling to room temperature, the suspension was filtered. The filter cake was washed with CH₃CN, collected and dried under vacuum to afford desired product 2-isothiocyanato-4-methoxybenzo[d]thiazole 4 (15.0 g, 100%). LC-MS [M+H]⁺ 223

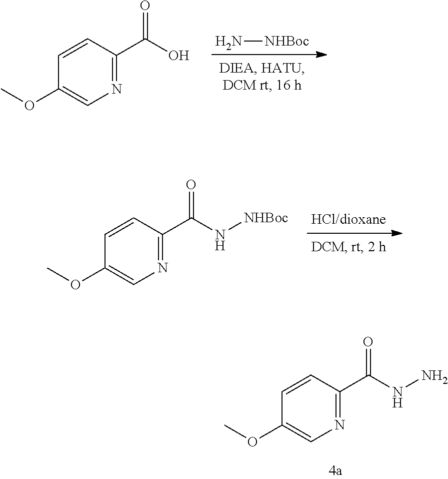 [SMILES: COc1ccc(C(=O)O)nc1 ; CC(C)(C)OC(=O)NN ; COc1ccc(C(=O)NNC(=O)OC(C)(C)C)nc1 ; COc1ccc(C(=O)NN)nc1]

### 5-methoxypicolinohydrazide (5a)

To a solution of 5-methoxypicolinic acid (300 mg, 2.0 mmol) in DCM (10 mL) was added DIPEA (516 mg, 4.0 mmol), BocNHNH₂ (397 mg, 3.0 mmol) and HATU (912 mg, 2.4 mmol). The resulting solution was stirred for 16 h at room temperature. After concentration, the residue was purified by column chromatography on silica gel eluted with PE/EA (1/1) to afford desired product tert-butyl 2-(5-methoxypicolinoyl)hydrazinecarboxylate (500 mg, 93%). LC-MS [M+H]⁺ 268

To a solution of compound tert-butyl 2-(5-methoxypicolinoyl)hydrazinecarboxylate (500 mg, 1.87 mmol) in DCM (20 mL) was added HCl/dioxane (20 mL) dropwise. After addition, the suspension was stirred for 2 h at room temperature. The mixture was concentrated to dryness to afford desired product 5-methoxypicolinohydrazide 5a (400 mg). LC-MS [M+H]⁺ 168

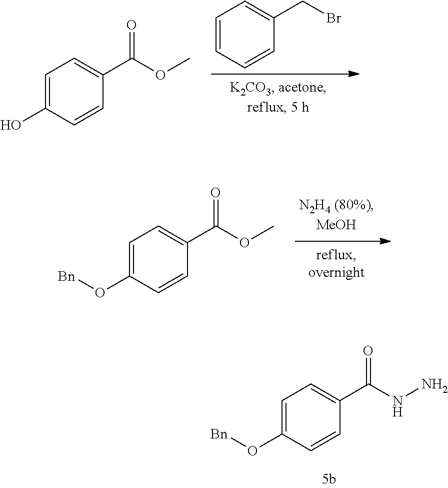

### 4-(benzyloxy)benzohydrazide (5b)

To a solution of methyl 4-hydroxybenzoate (25.0 g, 0.164 mol) in acetone (300 mL) was added K₂CO₃ (45.3 g, 0.328 mol) in portions, followed by BnBr (28.1 g, 0.164 mol) dropwise. After addition, the resulting mixture was refluxed for 5 h. After cooled to room temperature, the mixture was filtered through a pad of Celite and the filter cake was washed with EA. The combined filtrates was then evaporated. The residue was then diluted with MTBE (300 mL), washed with brine (2×100 mL), dried over Na₂SO₄ and concentrated to dryness to afford desired product methyl 4-(benzyloxy)benzoate (38.5 g, 97% yield). LC-MS [M+H]⁺ 243

To a solution of methyl 4-(benzyloxy)benzoate (40.0 g, 0.165 mol) in MeOH (300 mL) was added N₂H₄.H₂O (80%, 300 mL). The resulting solution was refluxed overnight. After cooled to room temperature, the mixture was concentrated. The resulting solid was dissolved in EA (1000 mL), washed with brine (2×300 mL), dried over Na₂SO₄ and concentrated to dryness to afford desired product 4-(benzyloxy)benzohydrazide 5b (35.0 g, 87.5%). LC-MS [M+H]⁺ 243

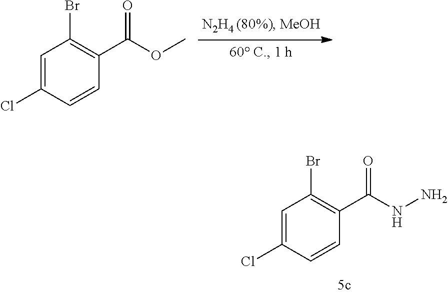

### 2-bromo-4-chlorobenzohydrazide (5c)

To a solution of methyl 2-bromo-4-chlorobenzoate (In-27, 5.0 g, 20.0 mmol) in MeOH (50 mL) was added N₂H₄.H₂O (80%, 50 mL). The resulting solution was heated at 60° C. for 1 h, until TLC show SM consumed. After concentration, the resulting solid was dissolved in EA (100 mL), washed with brine (2×30 mL), dried over Na₂SO₄ and concentrated to dryness to afford desired product 2-bromo-4-chlorobenzohydrazide 5c (2.8 g, 56%). ¹H NMR (300 MHz, DMSO-d₆): δ 9.58 (s, 1H), 7.80 (d, J=1.8 Hz, 1H), 7.52 (dd, J=8.1 Hz, J=2.1 Hz, 1H), 7.38 (d, J=8.1 Hz, 1H), 4.49 (s, 2H). LC-MS [M+H]⁺ 251;

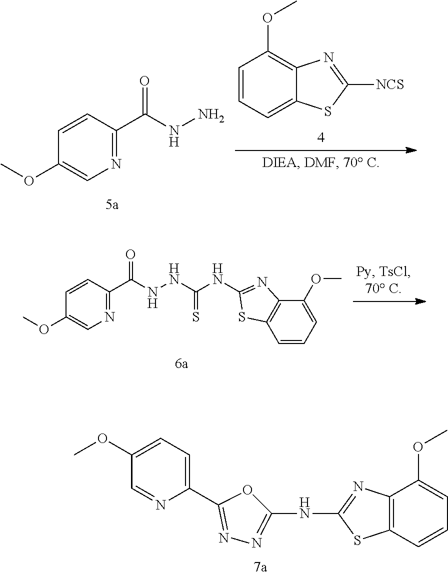

### N-(4-methoxybenzo[d]thiazol-2-yl)-5-(5-methoxypyridin-2-yl)-1,3,4-oxadiazol-2-amine (7a)

To a suspension of 5-methoxypicolinohydrazide 5a (150 mg, 0.6 mmol) in DMF (5 mL) was added DIPEA (310 mg, 2.4 mmol) and 2-isothiocyanato-4-methoxybenzo[d]thiazole 4 (133 mg, 0.6 mmol). The resulting solution was heated to 70° C. for 2 h. TLC showed the desired product N-(4-methoxybenzo[d]thiazol-2-yl)-2-(5-methoxypicolinoyl)hydrazinecarbothioamide 6a was formed. After cooling to room temperature, pyridine (142 mg, 1.8 mmol) and TsCl (172 mg, 0.9 mmol) was added and the solution was heated to 70° C. for 2 h. The reaction solution was filtered and the filtrate was concentrated and purified by prep-TLC to afford the compound N-(4-methoxybenzo[d]thiazol-2-yl)-5-(5-methoxypyridin-2-yl)-1,3,4-oxadiazol-2-amine 7a. ¹H NMR (300 MHz, DMSO-d₆): δ 8.45 (s, 1H), 8.44-8.08 (d, 1H), 7.61-7.57 (dd, 1H), 7.45-7.42 (d, 1H), 7.26-7.20 (t, 1H), 7.10-7.07 (d, 1H), 3.94 (s, 3H), 3.93 (s, 3H). LCMS (TOF-ESI) for C₁₆H₁₃N₅O₃S, calculated for [M+H]: 356.0809, found for [M+H]⁺:356.0809.

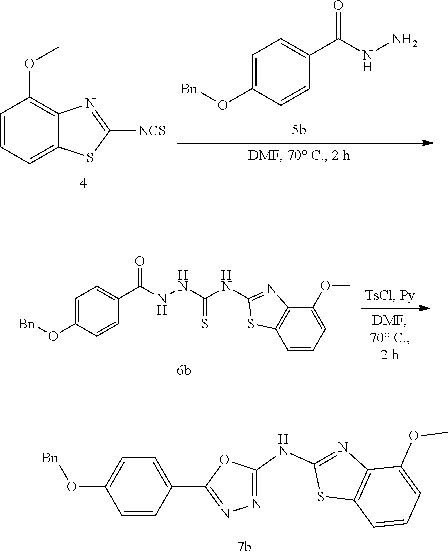

### 5-(4-(benzyloxy)phenyl)-N-(4-methoxybenzo[d]thiazol-2-yl)-1,3,4-oxadiazol-2-amine (7b)

To a suspension of 2-isothiocyanato-4-methoxybenzo[d]thiazole 4 (15.0 g, crude, 60.0 mmol) in DMF (150 mL) was added DIPEA (21 mL, 15.5 g, 120.0 mmol) dropwise. The suspension turned homogeneous and brown. Then 4-(benzyloxy)benzohydrazide 5b (15.3 g, 63.0 mmol) was added in portions and the resulting solution was heated to 70° C. for 2 h. LCMS showed the starting material was consumed completely and desired product 2-(4-(benzyloxy)benzoyl)-N-(4-methoxybenzo[d]thiazol-2-yl)hydrazinecarbothioamide 6b was formed. LC-MS [M+H]⁺ 431; After cooling to room temperature, pyridine (14.2 g, 180.0 mmol) and TsCl (17.2 g, 90.0 mmol) was added in portions and the solution was heated to 70° C. for 2 h. The above solution was concentrated under vacuum. The residue was added MeOH (100 mL) and stirred for 10 min. The resulting suspension was filtered. The filter cake was washed with MeOH, EA and TBME in turn. The filter cake was collected and dried to afford the desired product 5-(4-(benzyloxy)phenyl)-N-(4-methoxybenzo[d]thiazol-2-yl)-1,3,4-oxadiazol-2-amine 7b (9.0 g, 35% yield). LCMS (TOF-ESI) for C₂₃H₁₈N₄O₃S, calculated for [M+H]: 431.1170, found for [M+H]⁺:

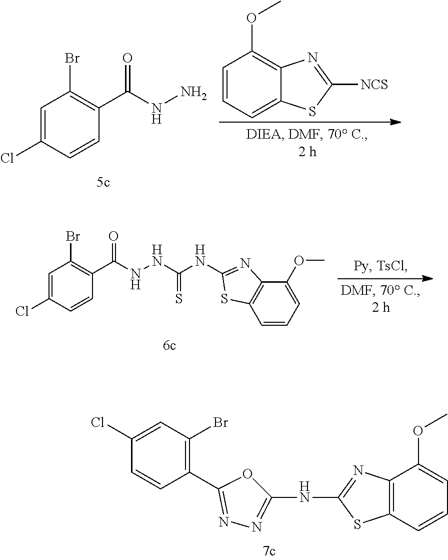

### 5-(2-bromo-4-chlorophenyl)-N-(4-methoxybenzo[d]thiazol-2-yl)-1,3,4-oxadiazol-2-amine (7c)

To a suspension of 2-bromo-4-chlorobenzohydrazide 5c (200 mg, 0.8 mmol) in DMF (4 mL) was added 2-isothiocyanato-4-methoxybenzo[d]thiazole 4 (178 mg, 0.8 mmol) and DIPEA (206 mg, 1.6 mmol). The suspension turned homogeneous immediately. The resulting solution was heated to 70° C. for 2 h. LCMS showed the desired product 2-(2-bromo-4-chlorobenzoyl)-N-(4-methoxybenzo[d]thiazol-2-yl)hydrazinecarbothioamide was formed. After cooling to room temperature, pyridine (190 mg, 2.4 mmol) and TsCl (229 mg, 1.2 mmol) was added and the solution was heated to 70° C. for 2 h. The reaction solution was filtered and the filtrate was directly purified by silica-gel chromatography to afford the product 5-(2-bromo-4-chlorophenyl)-N-(4-methoxybenzo[d]thiazol-2-yl)-1,3,4-oxadiazol-2-amine 7c. ¹H NMR (300 MHz, DMSO-d₆): δ 8.03-8.02 (d, 1H), 7.94-7.91 (d, 1H), 7.70-7.66 (dd, 1H), 7.46-7.43 (d, 1H), 7.27-7.21 (t, 1H), 7.11-7.08 (d, 1H), 3.94 (s, 3H). LCMS (TOF-ESI) for C₁₆H₁₀BrClN₄O₂S, calculated for [M+H]: 436.9466, found for [M+H]⁺: 436.9469.

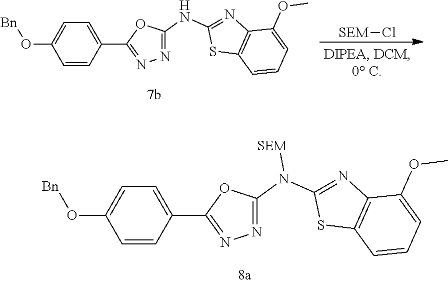 [SMILES: COc1cccc2sc(Nc3nnc(-c4ccc(OCc5ccccc5)cc4)o3)nc12 ; C[Si](C)(C)CCOCCl ; COc1cccc2sc(N(COCC[Si](C)(C)C)c3nnc(-c4ccc(OCc5ccccc5)cc4)o3)nc12]

### 5-(4-(benzyloxy)phenyl)-N-(4-methoxybenzo[d]thiazol-2-yl)-N-((2-(trimethylsilyl)ethoxy)methyl)-1,3,4-oxadiazol-2-amine (8a)

To a solution of 5-(4-(benzyloxy)phenyl)-N-(4-methoxybenzo[d]thiazol-2-yl)-1,3,4-oxadiazol-2-amine 7b (8.0 g, 18.6 mmol) in DCM (200 mL) cooled at 0° C. was added DIPEA (4.8 g, 37.2 mmol) dropwise, followed by SEM-Cl (3.7 g, 22.3 mmol) in DCM (30 mL) dropwise. After addition, the resulting solution was stirred for 2 h at 0° C. The mixture was concentrated, the residue was dissolved in EA (300 mL), washed with brine (3×100) NRcRd, —S(O)2NRcR₂SO₄ and concentrated to dryness. The crude product was purified by flash column chromatography on silica gel to give a crude product 5-(4-(benzyloxy)phenyl)-N-(4-methoxybenzo[d]thiazol-2-yl)-N-((2-(trimethylsilyl)ethoxy)methyl)-1,3,4-oxadiazol-2-amine 8a (9.0 g crude, 86.5% yield). LCMS (TOF-ESI) for C₂₉H₃₂N₄O₄SSi, calculated for [M+H]: 561.1984, found for [M+H]⁺: 561.

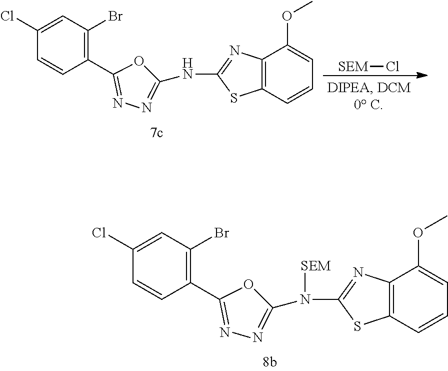 [SMILES: COc1cccc2sc(Nc3nnc(-c4ccc(Cl)cc4Br)o3)nc12 ; C[Si](C)(C)CCOCCl ; COc1cccc2sc(N(COCC[Si](C)(C)C)c3nnc(-c4ccc(Cl)cc4Br)o3)nc12]

### 5-(2-bromo-4-chlorophenyl)-N-(4-methoxybenzo[d]thiazol-2-yl)-N-((2-(trimethylsilyl)ethoxy)methyl)-1,3,4-oxadiazol-2-amine (8b)

To a solution of 5-(2-bromo-4-chlorophenyl)-N-(4-methoxybenzo[d]thiazol-2-yl)-1,3,4-oxadiazol-2-amine 7c (1.0 g, 2.3 mmol) in DCM (30 mL) cooled at 0° C. was added DIPEA (442 mg, 3.4 mmol) dropwise, followed by SEM-Cl (419 mg, 2.5 mmol) in DCM (10 mL) dropwise. After addition, the resulting solution was stirred for 2 h at 0° C. TLC show completion of the reaction. After concentration, the residue was dissolved in EA (100 mL), washed with brine (3×30 mL), dried over Na₂SO₄ and concentrated to dryness to give a crude product 5-(2-bromo-4-chlorophenyl)-N-(4-methoxybenzo[d]thiazol-2-yl)-N-((2-(trimethylsilyl)ethoxy)methyl)-1,3,4-oxadiazol-2-amine 8b (800 mg, 61% yield). LCMS (TOF-ESI) for C₂₂H₂₄BrClN₄O₃SSi, calculated for [M+H]: 568.5680, found for [M+H]⁺:569.

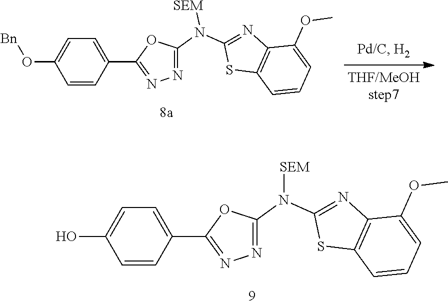 [SMILES: COc1cccc2sc(N(COCC[Si](C)(C)C)c3nnc(-c4ccc(OCc5ccccc5)cc4)o3)nc12 ; COc1cccc2sc(N(COCC[Si](C)(C)C)c3nnc(-c4ccc(O)cc4)o3)nc12]

### 4-(5-((4-methoxybenzo[d]thiazol-2-yl)((2-(trimethylsilyl)ethoxy)methyl)amino)-1,3,4-oxadiazol-2-yl)phenol (9)

To a solution of 5-(4-(benzyloxy)phenyl)-N-(4-methoxybenzo[d]thiazol-2-yl)-N-((2-(trimethylsilyl)ethoxy)methyl)-1,3,4-oxadiazol-2-amine 8a (9.0 g, crude, 16.1 mmol) in a mixed solution of THF/MeOH (1000 mL, V/V=1/1) was added wet Pd/C (50 g). The mixture was degassed and purged with H₂ several times, and then stirred overnight under H₂ (balloon). After reaction was completed, the mixture was filtered through a pad of celite and the filter cake was washed with a mixed solution of DCM/MeOH (1000 mL, V/V=1/1). The combined filtrate was concentrated to dryness to afford the desired product 4-(5-((4-methoxybenzo[d]thiazol-2-yl)((2-(trimethylsilyl)ethoxy)methyl)amino)-1,3,4-oxadiazol-2-yl)phenol 9 (4.2 g, crude, 55.6% yield). LCMS (TOF-ESI) for C₂₂H₂₆N₄O₄SSi, calculated for [M+H]: 471.1514, found [M+H]⁺:471.

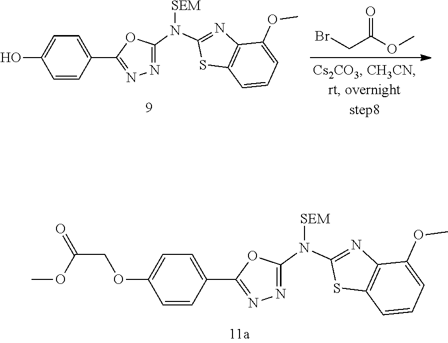

### methyl 2-(4-(5-((4-methoxybenzo[d]thiazol-2-yl)((2-(trimethylsilyl)ethoxy)methyl)amino)-1,3,4-oxadiazol-2-yl)phenoxy)acetate (11a)

To a solution of 4-(5-((4-methoxybenzo[d]thiazol-2-yl)((2-(trimethylsilyl)ethoxy)methyl)amino)-1,3,4-oxadiazol-2-yl)phenol 9 (2.5 g, crude, 5.3 mmol) in CH₃CN (100 mL) was added Cs₂CO₃ (3.5 g, 10.6 mmol), followed by methyl 2-bromoacetate (0.98 g, 6.4 mmol) dropwise. After addition, the resulting suspension was stirred overnight at room temperature. The mixture was concentrated and the residue was partitioned between EA (100 mL) and water (50 mL). The organic layer was washed with brine (2×50 mL), dried over Na₂SO₄ and concentrated to dryness to afford crude product methyl 2-(4-(5-((4-methoxybenzo[d]thiazol-2-yl)((2-(trimethylsilyl)ethoxy)methyl)amino)-1,3,4-oxadiazol-2-yl)phenoxy)acetate 11a (2.8 g crude, 97% yield). LCMS (TOF-ESI) for C₂₅H₃₀N₄O₆SSi, calculated for [M+H]: 543.1725, found for [M+H]⁺: 543.

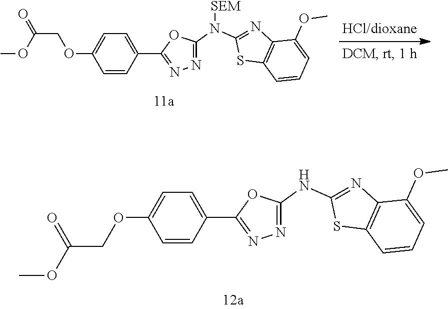

### methyl 2-(4-(5-((4-methoxybenzo[d]thiazol-2-yl)amino)-1,3,4-oxadiazol-2-yl)phenoxy)acetate (12a)

To a solution of methyl 2-(4-(5-((4-methoxybenzo[d]thiazol-2-yl)((2-(trimethylsilyl)ethoxy)methyl)amino)-1,3,4-oxadiazol-2-yl)phenoxy)acetate 11a (2.8 g, crude, 5.2 mmol) in DCM (30 mL) was added a solution of HCl/dioxane (30 mL) dropwise. After addition, the resulting solution was stirred for 1 h at room temperature. The suspension was filtered. The filter cake was washed with DCM, collected and dried under vacuum to afford desired product compound methyl 2-(4-(5-((4-methoxybenzo[d]thiazol-2-yl)amino)-1,3,4-oxadiazol-2-yl)phenoxy)acetate 12a (1.2 g, 56%). ¹H NMR (300 MHz, DMSO-d₆): δ 7.89-7.86 (d, 2H), 7.44-7.41 (d, 1H), 7.24-7.19 (t, 1H), 7.14-7.07 (m, 3H), 4.91 (s, 2H), 3.94 (s, 3H), 3.72 (s, 3H). LCMS (TOF-ESI) for C₁₉H₁₆N₄O₅S, calculated for [M+H]: 413.0911, found for [M+H]⁺:413.0920.

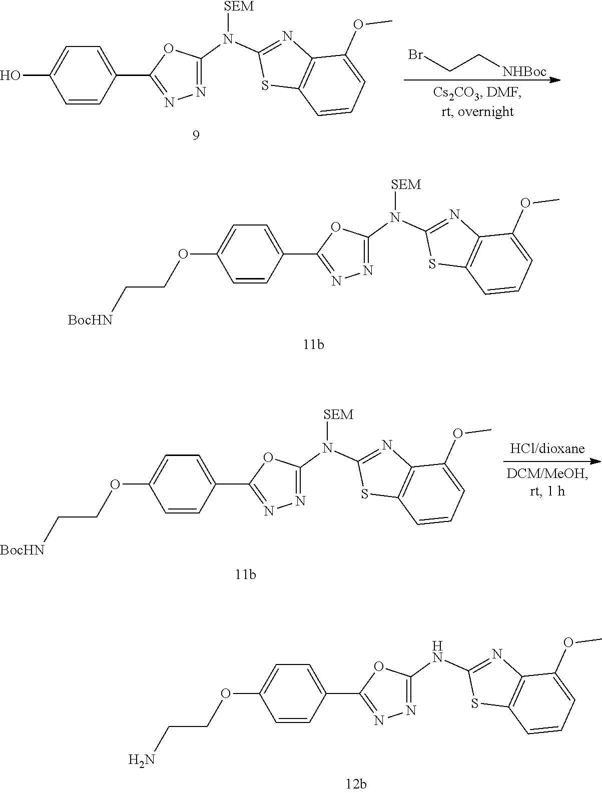

### 5-(4-(2-aminoethoxy)phenyl)-N-(4-methoxybenzo[d]thiazol-2-yl)-1,3,4-oxadiazol-2-amine (12b)

To a solution of 4-(5-((4-methoxybenzo[d]thiazol-2-yl)((2-(trimethylsilyl)ethoxy)methyl)amino)-1,3,4-oxadiazol-2-yl)phenol 9 (2.5 g, crude, 5.3 mmol) in DMF (40 mL) was added Cs₂CO₃ (3.5 g, 10.6 mmol), followed by tert-butyl 2-bromoethylcarbamate (1.4 g, 6.4 mmol). After addition, the resulting mixture was stirred overnight at room temperature. The mixture was concentrated and the residue was partitioned between EA (100 mL) and water (50 mL). The organic layer was washed with brine (2×50 mL), dried over Na₂SO₄ and concentrated to dryness to afford crude product tert-butyl (2-(4-(5-((4-methoxybenzo[d]thiazol-2-yl)((2-(trimethylsilyl)ethoxy)methyl)amino)-1,3,4-oxadiazol-2-yl)phenoxy)ethyl)carbamate 11b.

The crude tert-butyl(2-(4-(5-((4-methoxybenzo[d]thiazol-2-yl)((2-(trimethylsilyl)ethoxy)methyl)amino)-1,3,4-oxadiazol-2-yl)phenoxy)ethyl)carbamate 11b was then dissolved in a mixed solution of DCM/MeOH (30 mL, V/V=10/1) and HCl/dioxane (30 mL) was added dropwise. The solution was stirred for 1 h at room temperature. The suspension was filtered. The filter cake was collected and dried to afford the compound 5-(4-(2-aminoethoxy)phenyl)-N-(4-methoxybenzo[d]thiazol-2-yl)-1,3,4-oxadiazol-2-amine 12b (1.1 g, 50% over 2 steps). ¹H NMR (300 MHz, DMSO-d₆): δ 7.94-7.91 (d, 2H), 7.44-7.41 (d, 1H), 7.25-7.20 (t, 1H), 7.19-7.16 (d, 2H), 7.10-7.08 (d, 1H), 4.28-4.24 (m, 2H), 3.95 (s, 3H), 3.28-3.27 (m, 2H). LCMS (TOF-ESI) for C₁₈H₁₇N₅O₃S, calculated for [M+H]: 384.1122, found for [M+H]⁺: 384.1130.

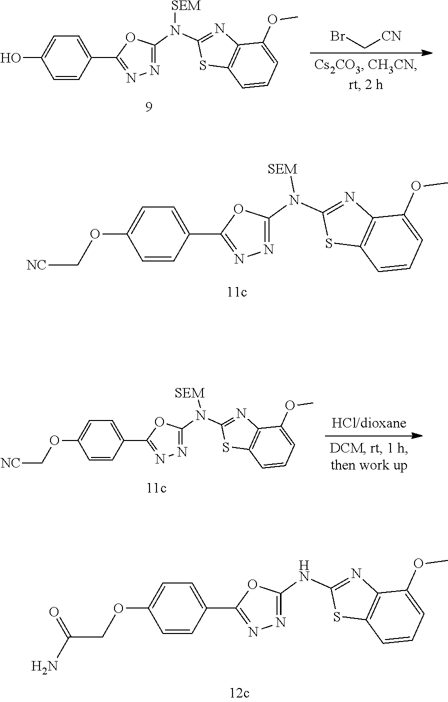

### 2-(4-(5-((4-methoxybenzo[d]thiazol-2-yl)amino)-1,3,4-oxadiazol-2-yl)phenoxy)acetamide (12c)

To a solution of compound 4-(5-((4-methoxybenzo[d]thiazol-2-yl)((2-(trimethylsilyl)ethoxy)methyl)amino)-1,3,4-oxadiazol-2-yl)phenol 9 (500 mg, crude, 1.06 mmol) in CH₃CN (20 mL) was added Cs₂CO₃ (691 mg, 2.12 mmol), followed by 2-bromoacetonitrile (153 mg, 1.27 mmol) dropwise. After addition, the resulting solution was stirred for 2 h at room temperature. The mixture was concentrated and the residue was partitioned between EA (50 mL) and water (20 mL). The organic layer was washed with brine (2×20 mL), dried over Na₂SO₄ and concentrated to dryness to afford crude product 2-(4-(5-((4-methoxybenzo[d]thiazol-2-yl)((2-(trimethylsilyl)ethoxy)methyl)amino)-1,3,4-oxadiazol-2-yl)phenoxy)acetonitrile 11c.

The crude 2-(4-(5-((4-methoxybenzo[d]thiazol-2-yl)((2-(trimethylsilyl)ethoxy)methyl)amino)-1,3,4-oxadiazol-2-yl)phenoxy)acetonitrile 11c was then taken in DCM (10 mL) and HCl/dioxane (10 mL) was added dropwise. The resulting solution was stirred for 1 h at room temperature. The mixture was concentrated. To the obtained residue was added H₂O (10 mL), basified with NaHCO₃ to pH˜4 and extracted with EA (2×30 mL). The organic layers were combined, concentrated and purified with prep-TLC to afford the desired product compound 2-(4-(5-((4-methoxybenzo[d]thiazol-2-yl)amino)-1,3,4-oxadiazol-2-yl)phenoxy)acetamide 12c (30.1 mg). III NMR (300 MHz, DMSO-d₆): δ 7.90-7.87 (d, 2H), 7.43-7.41 (d, 2H), 7.24-7.19 (t, 1H), 7.14-7.11 (d, 2H), 7.09-7.06 (d, 1H), 4.53 (s, 2H), 3.94 (s, 3H). LCMS (TOF-ESI) for C₁₈H₁₅N₅O₄S, calculated for [M+H]: 398.0915, found for [M+H]⁺: 398.0920.

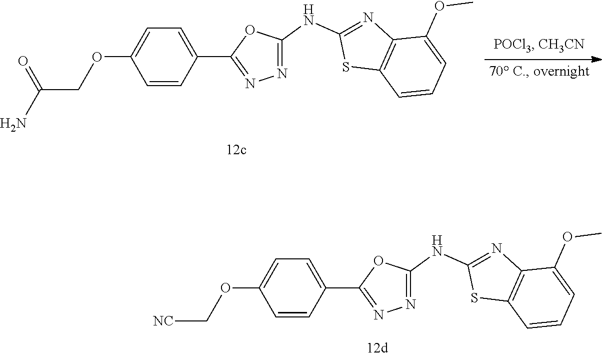 [SMILES: COc1cccc2sc(Nc3nnc(-c4ccc(OCC(N)=O)cc4)o3)nc12 ; COc1cccc2sc(Nc3nnc(-c4ccc(OCC#N)cc4)o3)nc12]

### 2-(4-(5-((4-methoxybenzo[d]thiazol-2-yl)amino)-1,3,4-oxadiazol-2-yl)phenoxy)acetonitrile (12d)

To a solution of compound 2-(4-(5-((4-methoxybenzo[d]thiazol-2-yl)amino)-1,3,4-oxadiazol-2-yl)phenoxy)acetamide 12c (300 mg, crude, 0.76 mmol) in CH₃CN (10 mL) was added POCl₃ (233 mg, 1.52 mmol). The resulting solution was stirred at 70° C. overnight. After cooled to room temperature, the mixture was concentrated and the residue was purified by prep-HPLC to afford the desired product compound 2-(4-(5-((4-methoxybenzo[d]thiazol-2-yl)amino)-1,3,4-oxadiazol-2-yl)phenoxy)acetonitrile 12d (22.4 mg). ¹H NMR (300 MHz, DMSO-d₆): δ 7.97-7.94 (d, 2H), 7.44-7.42 (d, 1H), 7.28-7.25 (d, 2H), 7.25-7.20 (t, 1H), 7.10-7.07 (d, 1H), 5.28 (s, 2H), 3.94 (s, 3H). LCMS (TOF-ESI) for C₁₈H₁₃N₅O₃S, calculated for [M+H]: 380.0809, found for [M+H]⁺: 380.0809.

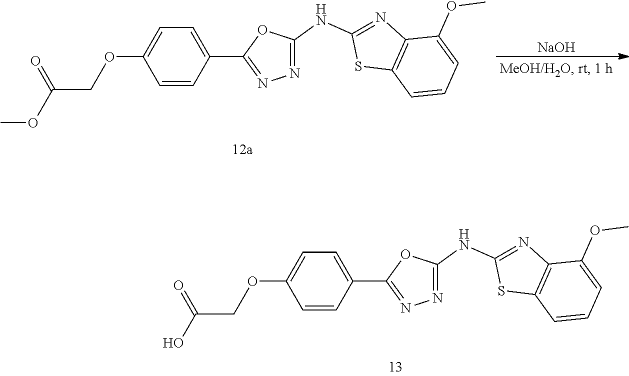

### 2-(4-(5-((4-methoxybenzo[d]thiazol-2-yl)amino)-1,3,4-oxadiazol-2-yl)phenoxy)acetic acid (13)

To a solution of compound methyl 2-(4-(5-((4-methoxybenzo[d]thiazol-2-yl)amino)-1,3,4-oxadiazol-2-yl)phenoxy)acetate 12a (150 mg, crude, 0.36 mmol) in MeOH/H₂O (3 mL, V/V=4/1) was added NaOH (44 mg, 1.1 mmol). The resulting solution was stirred for 1 h at room temperature. The solution was acidified with 2 N HCl to pH-3 and purified with prep-TLC to afford 69.2 mg pure product 2-(4-(5-((4-methoxybenzo[d]thiazol-2-yl)amino)-1,3,4-oxadiazol-2-yl)phenoxy)acetic acid 13. ¹H NMR (300 MHz, DMSO-d₆): δ 13.07 (br, 2H), 7.89-7.86 (d, 2H), 7.44-7.41 (d, 1H), 7.22 (t, 1H), 7.19-7.07 (m, 3H), 4.79 (s, 2H), 3.95 (s, 3H). LCMS (TOF-ESI) for C₁₈H₁₄N₄O₅S, calculated for [M+H]: 399.0755, found for [M+H]⁺: 399.0763.

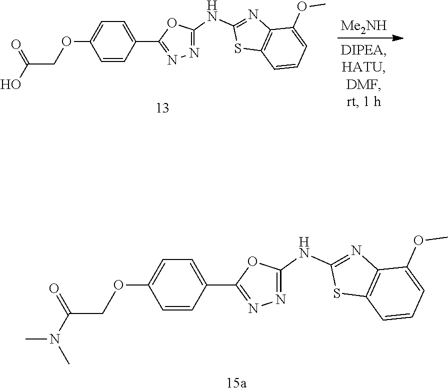

### 2-(4-(5-((4-methoxybenzo[d]thiazol-2-yl)amino)-1,3,4-oxadiazol-2-yl)phenoxy)-N,N-dimethylacetamide (15a)

To a solution of compound 2-(4-(5-((4-methoxybenzo[d]thiazol-2-yl)amino)-1,3,4-oxadiazol-2-yl)phenoxy)acetic acid 13 (150 mg, crude, 0.38 mmol) in DMF (3 mL) was added DIPEA (146 mg, 1.13 mmol), dimethylamine (0.57 mmol) and HATU (173 mg, 0.46 mmol). The resulting solution was stirred for 1 h at room temperature. After filtration the filtrate was purified by prep-TLC to afford pure products compound 2-(4-(5-((4-methoxybenzo[d]thiazol-2-yl)amino)-1,3,4-oxadiazol-2-yl)phenoxy)-N,N-dimethylacetamide 15a. ¹H NMR (300 MHz, DMSO-d₆): δ 7.87-7.84 (d, 2H), 7.44-7.41 (d, 1H), 7.25-7.19 (t, 1H), 7.10-7.07 (m, 3H), 4.93 (s, 2H), 3.95 (s, 3H), 3.01 (s, 3H), 2.86 (s, 3H). LCMS (TOF-ESI) for C₂₀H₁₉N₅O₄S, calculated for [M+H]: 426.1228, found for [M+H]⁺: 426.1238.

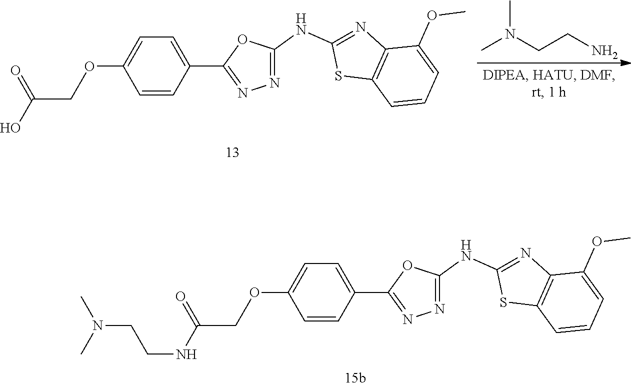

### N-(2-(dimethylamino)ethyl)-2-(4-(5-((4-methoxybenzo[d]thiazol-2-yl)amino)-1,3,4-oxadiazol-2-yl)phenoxy)acetamide (15b)

To a solution of compound 2-(4-(5-((4-methoxybenzo[d]thiazol-2-yl)amino)-1,3,4-oxadiazol-2-yl)phenoxy)acetic acid 13 (150 mg, crude, 0.38 mmol) in DMF (3 mL) was added DIPEA (146 mg, 1.13 mmol), N1,N1-dimethylethane-1,2-diamine (0.57 mmol) and HATU (173 mg, 0.46 mmol). The resulting solution was stirred for 1 h at room temperature. After filtration the filtrate was purified by prep-TLC to afford pure products compound N-(2-(dimethylamino)ethyl)-2-(4-(5-((4-methoxybenzo[d]thiazol-2-yl)amino)-1,3,4-oxadiazol-2-yl)phenoxy)acetamide 15b. ¹H NMR (300 MHz, DMSO-d₆): δ 9.34 (brs, 1H), 8.41-8.37 (t, 1H), 7.93-7.90 (d, 2H), 7.44-7.41 (d, 1H), 7.25-7.20 (t, 1H), 7.18-7.16 (d, 2H), 7.10-7.07 (d, 1H), 4.62 (s, 2H), 3.95 (s, 3H), 3.51-3.47 (m, 2H), 3.20 (m, 2H). 2.83 (s, 6H). LCMS (TOF-ESI) for C₂₂H₂₄N₆O₄S, calculated for [M+H]: 469.1650, found for [M+H]⁺: 469.1652.

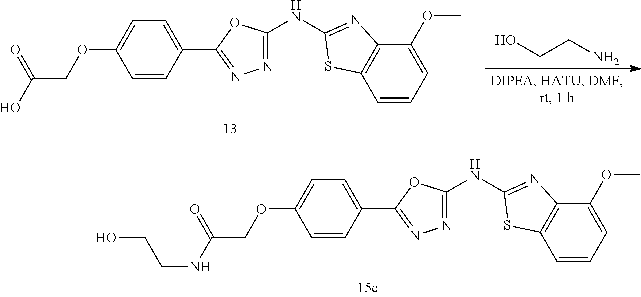

### N-(2-hydroxyethyl)-2-(4-(5-((4-methoxybenzo[d]thiazol-2-yl)amino)-1,3,4-oxadiazol-2-yl)phenoxy)acetamide (15c)

To a solution of compound 2-(4-(5-((4-methoxybenzo[d]thiazol-2-yl)amino)-1,3,4-oxadiazol-2-yl)phenoxy)acetic acid 13 (150 mg, crude, 0.38 mmol) in DMF (3 mL) was added DIPEA (146 mg, 1.13 mmol), 2-aminoethanol (0.57 mmol) and HATU (173 mg, 0.46 mmol). The resulting solution was stirred for 1 h at room temperature. After filtration the filtrate was purified by prep-TLC to afford pure products compound N-(2-hydroxyethyl)-2-(4-(5-((4-methoxybenzo[d]thiazol-2-yl)amino)-1,3,4-oxadiazol-2-yl)phenoxy)acetamide 15c. ¹H NMR (300 MHz, DMSO-d₆): δ 8.09 (t, 1H), 7.91-7.88 (d, 2H), 7.44-7.41 (d, 1H), 7.25-7.19 (t, 1H), 7.16-7.13 (d, 2H), 7.10-7.07 (d, 1H), 4.58 (s, 2H), 3.94 (s, 3H), 3.44-3.42 (t, 2H), 3.23-3.21 (q, 2H). LCMS (TOF-ESI) for C₂₀H₁₉N₅O₅S, calculated for [M+H]: 442.1177, found for [M+H]⁺:442.1188.

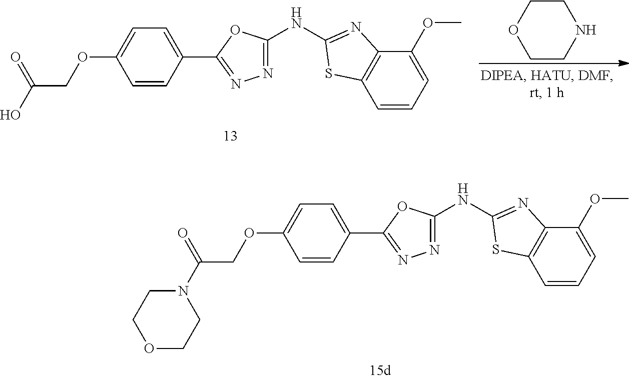 [SMILES: COc1cccc2sc(Nc3nnc(-c4ccc(OCC(=O)O)cc4)o3)nc12 ; C1COCCN1 ; COc1cccc2sc(Nc3nnc(-c4ccc(OCC(=O)N5CCOCC5)cc4)o3)nc12]

### 2-(4-(5-((4-methoxybenzo[d]thiazol-2-yl)amino)-1,3,4-oxadiazol-2-yl)phenoxy)-1-morpholinoethanone (15d)

To a solution of compound 2-(4-(5-((4-methoxybenzo[d]thiazol-2-yl)amino)-1,3,4-oxadiazol-2-yl)phenoxy)acetic acid 13 (150 mg, crude, 0.38 mmol) in DMF (3 mL) was added DIPEA (146 mg, 1.13 mmol), morpholine (0.57 mmol) and HATU (173 mg, 0.46 mmol). The resulting solution was stirred for 1 h at room temperature. After filtration the filtrate was purified by prep-TLC to afford pure products compound 2-(4-(5-((4-methoxybenzo[d]thiazol-2-yl)amino)-1,3,4-oxadiazol-2-yl)phenoxy)-1-morpholinoethanone 15d. ¹H NMR (300 MHz, DMSO-d₆): δ 7.88-7.85 (d, 2H), 7.44-7.41 (d, 1H), 7.25-7.19 (t, 1H), 7.12-7.07 (m, 3H), 4.96 (s, 2H), 3.94 (s, 3H), 3.62-3.58 (m, 4H), 3.47 (m, 4H). LCMS (TOF-ESI) for C₂₂H₂₁N₅O₅S, calculated for [M+H]: 468.1343, found for [M+H]⁺: 468.1345.

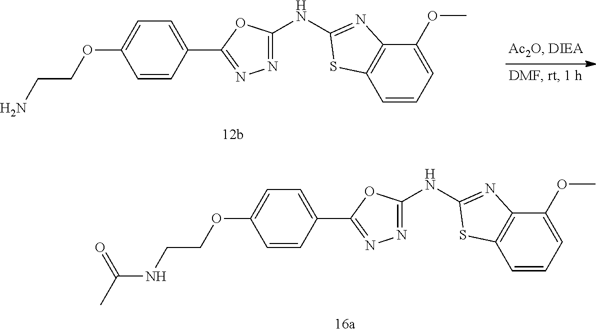

### N-(2-(4-(5-((4-methoxybenzo[d]thiazol-2-yl)amino)-1,3,4-oxadiazol-2-yl)phenoxy)ethyl)acetamide (16a)

To a solution of compound 5-(4-(2-aminoethoxy)phenyl)-N-(4-methoxybenzo[d]thiazol-2-yl)-1,3,4-oxadiazol-2-amine 12b (150 mg, crude, 0.39 mmol) in DMF (3 mL) was added DIPEA (151 mg, 1.17 mmol) and Ac₂O (51 mg, 0.50 mmol). The resulting solution was stirred for 1 h at room temperature. After filtration, the filtrate was purified by prep-TLC to afford pure products compound N-(2-(4-(5-((4-methoxybenzo[d]thiazol-2-yl)amino)-1,3,4-oxadiazol-2-yl)phenoxy)ethyl)acetamide 16a (12 mg). ¹H NMR (300 MHz, DMSO-d₆): δ 7.96-7.93 (d, 2H), 7.32-7.22 (m, 2H), 7.14-7.11 (d, 2H), 7.08-7.05 (d, 1H), 4.15-4.14 (t, 2H), 4.02 (s, 3H), 3.61 (t, 2H), 1.99 (s, 3H). LCMS (TOF-ESI) for C₂₀H₁₉N₅O₄S, calculated for [M+H]: 426.1228, found for [M+H]⁺: 426.1234.

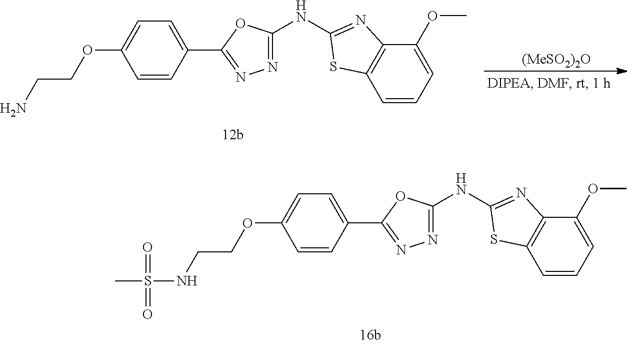

### N-(2-(4-(5-((4-methoxybenzo[d]thiazol-2-yl)amino)-1,3,4-oxadiazol-2-yl)phenoxy)ethyl)methanesulfonamide (16b)

To a solution of compound 5-(4-(2-aminoethoxy)phenyl)-N-(4-methoxybenzo[d]thiazol-2-yl)-1,3,4-oxadiazol-2-amine 12b (150 mg, crude, 0.39 mmol) in DMF (3 mL) was added DIPEA (151 mg, 1.17 mmol) and (MeSO₂)₂O (87 mg, 0.50 mmol). The resulting solution was stirred for 1 h at room temperature. After filtration, the filtrate was purified by prep-TLC to afford pure compound N-(2-(4-(5-((4-methoxybenzo[d]thiazol-2-yl)amino)-1,3,4-oxadiazol-2-yl)phenoxy)ethyl)methanesulfonamide 16b (22 mg). ¹H NMR (300 MHz, DMSO-d₆): δ 13.04 (brs, 1H), 7.90-7.87 (d, 2H), 7.44-7.41 (d, 1H), 7.33-7.30 (t, 1H), 7.25-7.19 (t, 1H), 7.15-7.12 (d, 2H), 7.09-7.07 (d, 1H), 4.15-4.11 (t, 2H), 3.94 (s, 3H), 3.38-3.36 (m, 2H), 2.96 (s, 3H). LCMS (TOF-ESI) for C₁₉H₁₉N₅O₅S₂, calculated for [M+H]: 462.0898, found for [M+H]⁺: 462.0902.

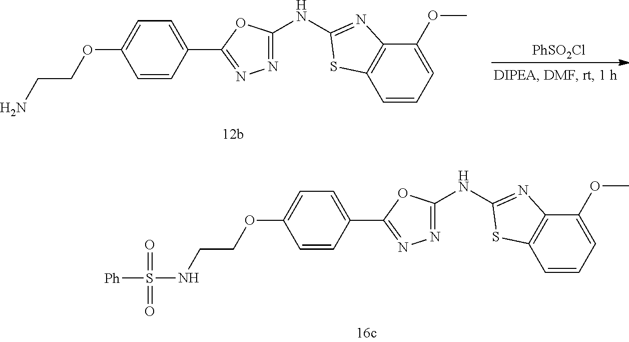 [SMILES: "PhSO2Cl" ; COc1cccc2sc(Nc3nnc(-c4ccc(OCCN)cc4)o3)nc12 ; COc1cccc2sc(Nc3nnc(-c4ccc(OCCNS(=O)(=O)c5ccccc5)cc4)o3)nc12]

### N-(2-(4-(5-((4-methoxybenzo[d]thiazol-2-yl)amino)-1,3,4-oxadiazol-2-yl)phenoxy)ethyl)benzenesulfonamide (16c)

To a solution of compound 5-(4-(2-aminoethoxy)phenyl)-N-(4-methoxybenzo[d]thiazol-2-yl)-1,3,4-oxadiazol-2-amine 12b (150 mg, crude, 0.39 mmol) in DMF (3 mL) was added DIPEA (151 mg, 1.17 mmol) and PhSO₂Cl (88 mg, 0.50 mmol). The resulting solution was stirred for 1 h at room temperature. After filtration, the filtrate was purified by prep-TLC twice to afford pure compound N-(2-(4-(5-((4-methoxybenzo[d]thiazol-2-yl)amino)-1,3,4-oxadiazol-2-yl)phenoxy)ethyl)benzenesulfonamide 16c (29 mg). ¹H NMR (300 MHz, DMSO-d₆): δ 13.04 (brs, 1H), 8.00-7.97 (t, 1H), 7.87-7.85 (m, 4H), 7.65-7.57 (m, 3H), 7.44-7.41 (d, 1H), 7.25-7.20 (t, 1H), 7.10-7.07 (d, 1H), 7.04-7.01 (d, 2H), 4.05-4.03 (m, 2H), 3.95 (s, 3H), 3.20-3.18 (m, 2H). LCMS (TOF-ESI) for C₂₄H₂₁N₅O₅S₂, calculated for [M+H]: 524.1054, found for [M+H]⁺: 524.1048.

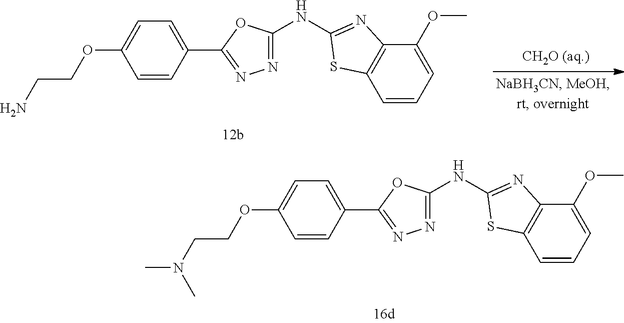

### 5-(4-(2-(dimethylamino)ethoxy)phenyl)-N-(4-methoxybenzo[d]thiazol-2-yl)-1,3,4-oxadiazol-2-amine (16d)

To a solution of compound 5-(4-(2-aminoethoxy)phenyl)-N-(4-methoxybenzo[d]thiazol-2-yl)-1,3,4-oxadiazol-2-amine 12b (150 mg, crude, 0.39 mmol) in MeOH (4 mL) was added DIPEA to adjust pH-7. Then CH₂O (0.5 mL), a drop of AcOH and NaBH₃CN (121 mg, 1.95 mmol) was added in turn. The resulting solution was stirred overnight at room temperature. After filtration, the filtrate was purified by prep-TLC to afford pure compound 5-(4-(2-(dimethylamino)ethoxy)phenyl)-N-(4-methoxybenzo[d]thiazol-2-yl)-1,3,4-oxadiazol-2-amine 16d (31 mg). ¹H NMR (300 MHz, DMSO-d₆): δ 8.02-7.99 (d, 2H), 7.32-7.21 (m, 4H), 7.08-7.06 (d, 1H), 4.48-4.46 (t, 2H), 4.03 (s, 3H), 3.68-3.65 (t, 2H), 3.03 (s, 6H). LCMS (TOF-ESI) for C₂₀H₂₁N₅O₃S, calculated for [M+H]: 412.1435, found for [M+H]⁺: 412.1436.

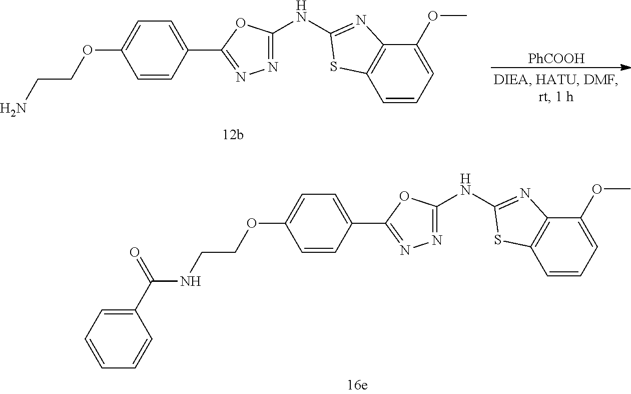 [SMILES: "PhCOOH" ; COc1cccc2sc(Nc3nnc(-c4ccc(OCCN)cc4)o3)nc12 ; COc1cccc2sc(Nc3nnc(-c4ccc(OCCNC(=O)c5ccccc5)cc4)o3)nc12]

### N-(2-(4-(5-((4-methoxybenzo[d]thiazol-2-yl)amino)-1,3,4-oxadiazol-2-yl)phenoxy)ethyl)benzamide (16e)

To a solution of compound 5-(4-(2-aminoethoxy)phenyl)-N-(4-methoxybenzo[d]thiazol-2-yl)-1,3,4-oxadiazol-2-amine 12b (150 mg, crude, 0.39 mmol) in DMF (3 mL) was added DIPEA (151 mg, 1.17 mmol), PhCO₂H (61 mg, 0.50 mmol) and HATU (190 mg, 0.50 mmol). The resulting solution was stirred for 1 h at room temperature. After filtration, the filtrate was purified by prep-TLC to afford compound 12 (30 mg, 93% purity), which was further purified by prep-TLC to afford pure compound N-(2-(4-(5-((4-methoxybenzo[d]thiazol-2-yl)amino)-1,3,4-oxadiazol-2-yl)phenoxy)ethyl)benzamide 16e (16 mg). ¹H NMR (300 MHz, DMSO-d₆): δ 8.72 (t, 1H), 7.89-7.86 (d, 4H), 7.53-7.41 (m, 4H), 7.24-7.14 (m, 3H), 7.09-7.06 (d, 1H), 4.24-4.21 (t, 2H), 3.94 (s, 3H), 3.68-3.67 (m, 2H). LCMS (TOF-ESI) for C₂₅H₂₁N₅O₄S, calculated for [M+H]: 488.1384, found for [M+H]⁺: 488.1394.

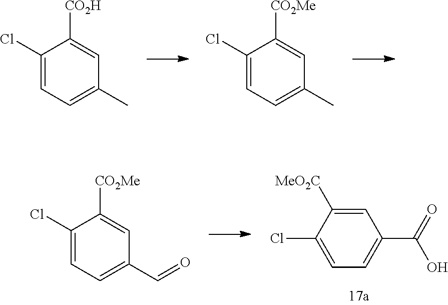

### Synthesis of 4-chloro-3-(methoxycarbonyl)benzoic acid (17a)

A solution of 2-chloro-5-methylbenzoic acid (5 g, 29.31 mmol, 1 equiv.) in MeOH (60 mL) was added with concentrated H₂SO₄ (1 mL), the mixture was refluxed for 20 h. Then cooled to room temperature and concentrated to remove most of MeOH, the residue was diluted with water and extracted with CH₂Cl₂, dried over Na₂SO₄, concentrated and purified via FCC (Hexanes:EtOAc, 10:1) to give product methyl 2-chloro-5-methylbenzoate as grey solid. A solution of above obtained methyl 2-chloro-5-methylbenzoate in CCl4 (60 mL) was added with NBS (13 g, 2.5 equiv.) and benzoyl peroxide (5 mol %, 0.4 g), the mixture was stirred at reflux condition for 48 h, then cooled to room temperature and filtered, the filtrated was collected and concentrated. The crude product was diluted with Et₂O and filtered again, the filtrate was collect and concentrated, which was used for next step without purification.

To a solution of crude product in acetone (50 mL) was added with AgNO₃ (11 g, 2.1 equiv., in water 6 mL), the mixture was stirred at 50° C. for 2 h. Then filtered, the filtrate was collect and concentrated to give crude product methyl 2-chloro-5-formylbenzoate. ¹H-NMR (600 MHz, CDCl₃): δ 10.04 (s, 1H), 8.59 (s, 1H), 8.16-8.15 (d, 1H), 7.62-7.60 (d, 1H), 4.00 (s, 3H).

To a solution of methyl 2-chloro-5-formylbenzoate (2 g, 10.07 mmol, 1 equiv.), NaH₂PO₄ (1.1 equiv., 1.4 g), 2-methyl-2-butene (1M, in THF, 5.6 mL) in THF/tBuOH/H₂O (20 mL/60 mL/16 mL) was added NaClO₂ (4.5 g, 5 equiv.) slowly at room temperature, the mixture was stirred for further 3 h. Then the HCl (1M) was added till pH-3 and extracted with EtOAc. The organic layer was concentrated to give product 4-chloro-3-(methoxycarbonyl)benzoic acid 17a as white solid. ¹H-NMR (600 MHz, CDCl₃): δ 8.59 (s, 1H), 8.16-8.15 (d, 1H), 7.62-7.60 (d, 1H), 3.99 (s, 3H); LCMS (TOF-ESI) for C₉H₇O₄Cl, Calculated for [M+H]: 215.0106; Found [M+H]⁺ for 215.0104.

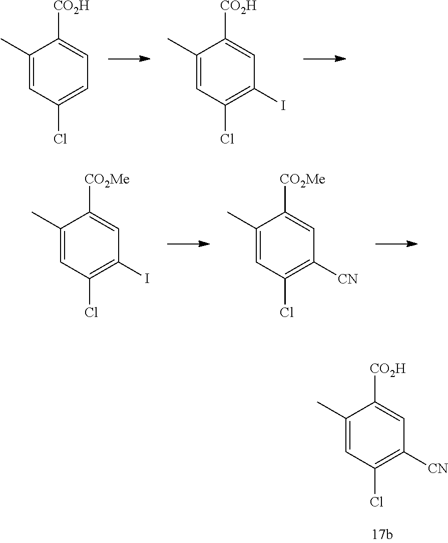

### Synthesis of 4-chloro-5-cyano-2-methylbenzoic acid (17b)

To a mixture of 4-chloro-2-methylbenzoic acid (2 g, 11.72 mmol, 1 equiv.) in concentrated H₂SO₄ (30 mL) was added with NIS (2.9 g, 1.1 equiv.) at 0° C. under Ar, the mixture was stirred at ice-bath temperature for 4 hour. After filtration, the cake was washed with water and dried under high vaccum to give crude product 4-chloro-5-iodo-2-methylbenzoic acid as grey pale solid. The obtained crude product 4-chloro-5-iodo-2-methylbenzoic acid was dissolved in MeOH (60 mL) and then added with concentrated H₂SO₄ (1 mL), the mixture was refluxed for 12 h. Then cooled to room temperature and concentrated to remove most of MeOH, the residue was diluted with water and extracted with CH₂Cl₂, dried over Na₂SO₄, concentrated and purified via FCC (Hexanes:EtOAc, 10:1) to give product methyl 4-chloro-5-iodo-2-methylbenzoate as grey solid 3.3 g (92% over 2 steps). ¹H-NMR (600 MHz, CDCl₃): δ 8.39 (s, 1H), 7.36 (s, 1H), 3.91 (s, 3H), 2.54 (s, 3H). LCMS (TOF-ESI) for C₉H₈ClIO₂, Calculated for [M+H]: 310.9331; Found [M+H]⁺ for 310.9328.

A suspension of methyl 4-chloro-5-iodo-2-methylbenzoate (2.2 g, 7.058 mmol, 1 equiv.) and CuCN (0.7 g, 1.1 equiv.) in DMF (4 mL) was stirred at 110° C. for 20 h under Ar. After cooled to room temperature and filtration, the filtrated was concentrated under high vacuum, the residue was purified via FCC (Hexanes:EtOAc, 10:1) to give product methyl 4-chloro-5-cyano-2-methylbenzoate as white solid 1.48 g (100%).¹H-NMR (600 MHz, CDCl₃): δ 8.26 (s, 1H), 7.44 (s, 1H), 3.94 (s, 3H), 2.68 (s, 3H); ¹³C NMR (125 MHz, CDCl₃): δ 164.8, 146.9, 139.3, 135.8, 132.6, 128.3, 114.8, 110.5, 52.1, 21.6. LCMS (TOF-ESI) for C₁₀H₈ClNO₂, Calculated for [M+H]: 210.0317; Found [M+H]⁺ for 210.0320.

To a solution of methyl 4-chloro-5-cyano-2-methylbenzoate (1.0 g, 4.77 mmol, 1 equiv.) in MeOH/THF (1:1, 50 mL) was added with LiOH (2M, in H₂O, 3 equiv., 7.2 mL), the mixture was stirred at room temperature for 12 h. After completion of reaction, the mixture was concentrated to remove volatile solvent. The residue was acidified by HCl (1M, pH=1), the precipitate was collected, washed with H₂O and dried under high vacuum to give product 4-chloro-5-cyano-2-methylbenzoic acid 17b as white solid. The product was dried under high vacuum and used for next step without further purification.

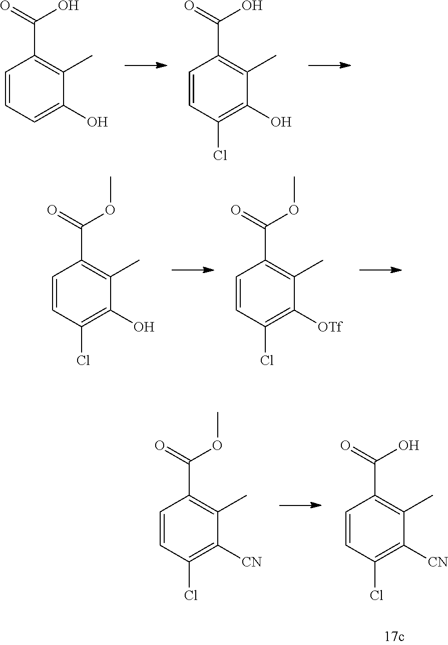 [SMILES: Cc1c(O)cccc1C(=O)O ; Cc1c(C(=O)O)ccc(Cl)c1O ; COC(=O)c1ccc(Cl)c(O)c1C ; COC(=O)c1ccc(Cl)c(OS(=O)(=O)C(F)(F)F)c1C ; COC(=O)c1ccc(Cl)c(C#N)c1C ; Cc1c(C(=O)O)ccc(Cl)c1C#N]

### Synthesis of 4-chloro-3-cyano-2-methylbenzoic acid (17c)

To a suspension of 3-hydroxy-2-methylbenzoic acid (25 g, 0.1644 mol, 1 equiv.) in H₂O (300 mL) was added with NaOH (3M, 62 mL, 1.1 equiv.), the solution was cooled to 0° C. in ice-bath, then a solution of aqueous NaClO (bleach, 10-15%, 1.5 equiv., 185 mL) was added through additional funnel in 1 h at 0° C. Upon completion of addition, the mixture was stirred for additional 10 mins, then HCl (3M, 100 mL, 1.8 equiv.) was added in one portion, the formed suspension was stirred for 1 h and then filtered. The cake was washed with water to afford product as pale pink solid, which was further recrystallized from Et₂O/Hexanes (3:1) to give product 4-chloro-3-hydroxy-2-methylbenzoic acid as pale solid. LCMS (TOF-ESI) for C₈H₇O₃Cl, Calculated for [M+H]: 187.0157; Found [M+H]⁺ for 187.0159.

A solution of 4-chloro-3-hydroxy-2-methylbenzoic acid (10 g, 53.76 mmol, 1 equiv.) in MeOH (100 mL) was added with concentrated H₂SO₄ (2 mL), the mixture was refluxed for 10 h. Then cooled to room temperature and concentrated to remove most of MeOH, the residue was diluted with water and extracted with CH₂Cl₂, dried over Na₂SO₄, concentrated and purified via FCC (Hexanes:EtOAc, 10:1) to give product methyl 4-chloro-3-hydroxy-2-methylbenzoate as grey solid. ¹H-NMR (600 MHz, d₆-DMSO): δ 12.94 (s, 1H), 9.41 (s, 1H), 7.28-7.24 (q, 2H), 2.39 (s, 3H); ¹³C NMR (125 MHz, d₆-DMSO): δ 168.5, 151.2, 131.3, 128.2, 126.4, 124.0, 121.7, 13.7; LCMS (TOF-ESI) for C₉H₉O₃Cl, Calculated for [M+H]: 201.0313; Found [M+H]⁺ for 201.0319.

To a solution of methyl 4-chloro-3-hydroxy-2-methylbenzoate (4.64 g, 23.197 mmol, 1 equiv.) and triethyl amine (8.1 mL, 2.5 equiv.) in CH₂Cl₂ (50 mL) was cooled to 0° C. with ice-bath, then Tf₂O (9 g, 1.3 equiv.) was added slowly, the mixture was slowly warmed up to room temperature and stirred for 3 h. The reaction mixture was concentrated and purified via FCC (Hexanes/EtOAc, 10:1) to provide product methyl 4-chloro-2-methyl-3-(((trifluoromethyl)sulfonyl)oxy)benzoate as white solid (5.4 g, 71%). LCMS (TOF-ESI) for C₁₀H₈ClF₃O₅S, Calculated for [M+H]: 332.9806; Found [M+H]⁺ for 332.9807.

A mixture of methyl 4-chloro-2-methyl-3-(((trifluoromethyl)sulfonyl)oxy)benzoate (5.4 g, 16.27 mmol, 1 equiv.), Pd(PPh₃)₄ (1 g, 5 mol %) and Zn(CN)₂ (1.3 g, 0.65 equiv.) in dry DMF (50 mL) was stirred at 100° C. under Ar for 12 h. Then cooled to room temperature, the solvent was removed under high vacuum. The residue was extracted with Et₂O and the organic layer was separated, concentrated and purified via FCC (Hexanes/EtOAc, 15:1) to give desired product methyl 4-chloro-3-cyano-2-methylbenzoate (1.3 g, 40%). ¹H-NMR (600 MHz, CDCl₃): δ 8.05-8.04 (d, 1H), 7.44-7.43 (d, 1H), 3.95 (s, 3H), 2.86 (s, 3H); ¹³C NMR (125 MHz, CDCl₃): δ 165.4, 145.7, 140.4, 134.6, 128.9, 126.7, 115.6, 114.3, 52.1, 19.5. LCMS (TOF-ESI) for C₁₀H₈ClNO₂, Calculated for [M+H]: 210.0317; Found [M+H]⁺ for 210.0316.

To a solution of methyl 4-chloro-3-cyano-2-methylbenzoate (1.3 g, 6.22 mmol, 1 equiv.) in MeOH/THF (1:1, 50 mL) was added with LiOH (2M, in H₂O, 15 mL), the mixture was stirred at room temperature for 12 h. After completion of reaction, the mixture was concentrated to remove volatile solvent. The residue was acidified by HCl (1M, pH=1), the precipitate was collected, washed with H₂O and dried under high vaccum to give product 4-chloro-3-cyano-2-methylbenzoic acid 17c as white solid. LCMS (TOF-ESI) for C₉H₆ClNO₂, Calculated for [M+H]: 196.0160; Found [M+H]⁺ for 196.0165.

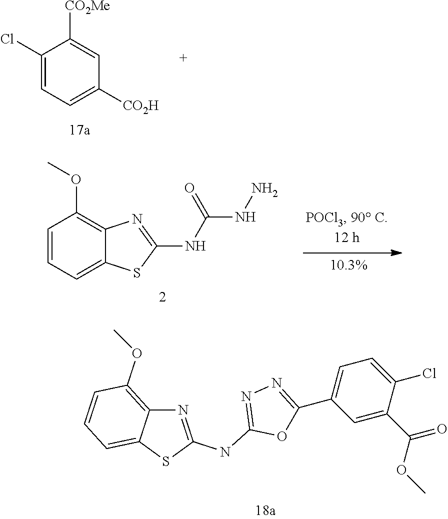 [SMILES: COC(=O)c1cc(C(=O)O)ccc1Cl ; COc1cccc2sc(NC(=O)NN)nc12 ; COC(=O)c1cc(-c2nnc(Nc3nc4c(OC)cccc4s3)o2)ccc1Cl]

### Methyl 2-chloro-5-(5-((4-methoxybenzo[d]thiazol-2-yl)amino)-1,3,4-oxadiazol-2-yl) benzoate (18a)

A mixture of 4-chloro-3-(methoxycarbonyl)benzoic acid 17a (0.5 g, 2.33 mmol, 1 equiv.) and N-(4-methoxybenzo[d]thiazol-2-yl)hydrazinecarboxamide 2 (0.56 g, 1 equiv.) in POCl₃ (10 mL) was stirred at 90° C. under Ar for 12 h. After cooled to room temperature, the reaction mixture was poured onto ice, the mixture was slowly warmed up to room temperature and filtered. The cake was collected and washed with water and subjected to FCC (Hexanes:EtOAc, 2:1) to give product as yellow solid, which was further recrystallized from MeOH to yield weak pink solid methyl 2-chloro-5-(5-((4-methoxybenzo[d]thiazol-2-yl)amino)-1,3,4-oxadiazol-2-yl) benzoate 18a (0.1 g, 10.3%). ¹H-NMR (600 MHz, CDCl₃): δ 8.55 (s, 1H), 8.11-8.09 (d, 1H), 7.62-7.60 (d, 1H), 7.26-7.23 (t, 1H), 7.19-7.17 (d, 1H), 6.93-6.91 (d, 1H), 4.00 (s, 3H), 3.99 (s, 3H), 3.51 (s, 1H); ¹³C NMR (125 MHz, CDCl₃): δ 165.2, 164.6, 158.1, 145.6, 136.0, 131.5, 130.1, 129.1, 128.7, 124.5, 122.7, 113.6, 107.7, 55.4, 52.2. LCMS (TOF-ESI) for C₁₈H₁₃N₄O₄ClS, Calculated for [M+H]: 417.0419; Found [M+H]⁺ for 417.0418.

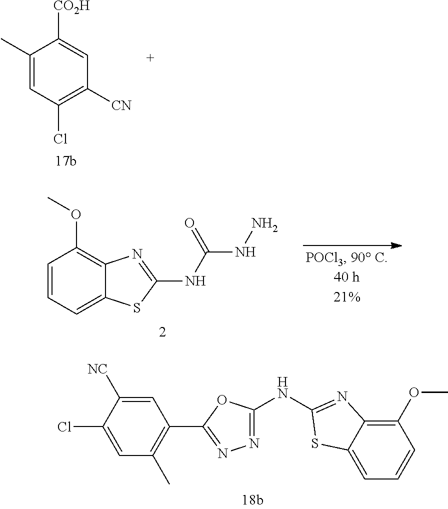 [SMILES: Cc1cc(Cl)c(C#N)cc1C(=O)O ; COc1cccc2sc(NC(=O)NN)nc12 ; COc1cccc2sc(Nc3nnc(-c4cc(C#N)c(Cl)cc4C)o3)nc12]

### 2-chloro-5-(5-((4-methoxybenzo[d]thiazol-2-yl)amino)-1,3,4-oxadiazol-2-yl)-4-methylbenzonitrile (18b)

A solution of 4-chloro-5-cyano-2-methylbenzoic acid 17b (0.3 g, 1.54 mmol, 1 equiv.) in POCl₃ (10 mL) was stirred at 90° C. under Ar for 1 h, then cooled to room temperature and N-(4-methoxybenzo[d]thiazol-2-yl)hydrazinecarboxamide 2 (0.38 g, 1 equiv.) was added. The mixture was stirred for 12 h at 90° C. under Ar. After cooled to room temperature, the reaction mixture was concentrated under high vacuum. The residue was treated with Et₃N and then concentrated and washed with water and subjected to FCC (CH₂Cl₂/MeOH, 20:1) to give product as light grey solid 2-chloro-5-(5-((4-methoxybenzo[d]thiazol-2-yl)amino)-1,3,4-oxadiazol-2-yl)-4-methylbenzonitrile 18b (0.13 g, 21%). ¹H-NMR (600 MHz, d₆-CDCl₃): δ 8.28 (s, 1H), 7.53 (s, 1H), 7.26-7.25 (t, 1H), 7.20-7.19 (d, 1H), 6.94-6.93 (d, 1H), 4.01 (s, 3H), 2.85 (s, 3H); ¹³C NMR (125 MHz, d₆-CDCl₃): δ 157.7, 144.7, 136.4, 133.3, 132.9, 123.9, 123.4, 115.4, 114.5, 110.1, 108.9, 56.0, 21.7; LCMS (TOF-ESI) for C₁₈H₁₂ClN₅O₂S, Calculated for [M+H]: 398.0473; Found [M+H]⁺ for 398.0479.

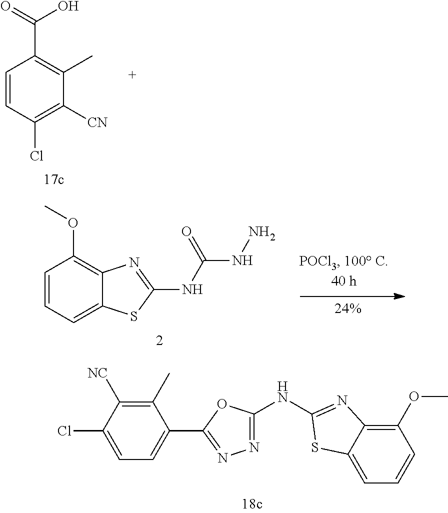 [SMILES: Cc1c(C(=O)O)ccc(Cl)c1C#N ; COc1cccc2sc(NC(=O)NN)nc12 ; COc1cccc2sc(Nc3nnc(-c4ccc(Cl)c(C#N)c4C)o3)nc12]

### 6-chloro-3-(5-((4-methoxybenzo[d]thiazol-2-yl)amino)-1,3,4-oxadiazol-2-yl)-2-methylbenzonitrile (18c)

A solution of 4-chloro-3-cyano-2-methylbenzoic acid 17c (0.3 g, 1.54 mmol, 1 equiv.) in POCl₃ (10 mL) was stirred at 90° C. under Ar for 1 h, then cooled to room temperature and N-(4-methoxybenzo[d]thiazol-2-yl)hydrazinecarboxamide 2 (0.38 g, 1 equiv.) was added. The mixture was stirred for 12 h at 90° C. under Ar. After cooled to room temperature, the reaction mixture was concentrated under high vacuum. The residue was treated with Et₃N and then concentrated and washed with water and subjected to FCC (CH₂Cl₂/MeOH, 20:1) to give product as light grey solid 6-chloro-3-(5-((4-methoxybenzo[d]thiazol-2-yl)amino)-1,3,4-oxadiazol-2-yl)-2-methylbenzonitrile 18c (0.15 g, 24%). ¹H-NMR (600 MHz, d₆-DMSO): δ 8.10-8.08 (d, 1H), 7.70-7.68 (d, 1H), 7.36-7.35 (d, 1H), 7.21-7.18 (t, 1H), 7.04-7.03 (d, 1H), 3.94 (s, 3H), 2.87 (s, 3H); ¹³C NMR (125 MHz, d₆-DMSO): δ 157.9, 142.9, 137.5, 133.1, 127.8, 123.8, 123.4, 114.9, 114.7, 114.4, 108.7, 55.9, 20.2; LCMS (TOF-ESI) for C₁₅H₁₂ClN₅O₂S, Calculated for [M+H]: 398.0473; Found [M+H]⁺ for 398.0478.

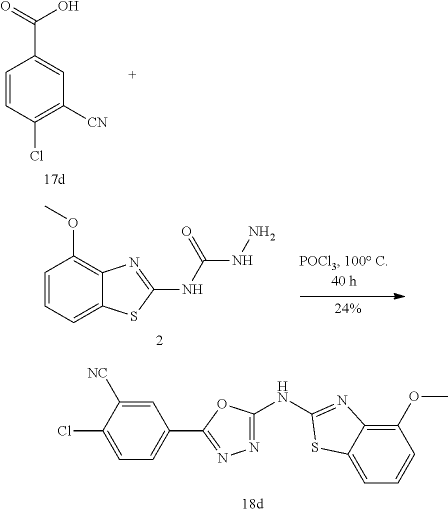 [SMILES: N#Cc1cc(C(=O)O)ccc1Cl ; COc1cccc2sc(NC(=O)NN)nc12 ; COc1cccc2sc(Nc3nnc(-c4ccc(Cl)c(C#N)c4)o3)nc12]

### 2-chloro-5-(5-((4-methoxybenzo[d]thiazol-2-yl)amino)-1,3,4-oxadiazol-2-yl)benzonitrile (18d)

A solution of 4-chloro-3-cyanobenzoic acid 17d (0.3 g, 1.657 mmol, 1 equiv.) in POCl₃ (10 mL) was stirred at 90° C. under Ar for 1 h, then cooled to room temperature and N-(4-methoxybenzo[d]thiazol-2-yl)hydrazinecarboxamide 2 (0.4 g, 1 equiv.) was added. The mixture was stirred for 12 h at 90° C. under Ar. After cooled to room temperature, the reaction mixture was concentrated under high vacuum. The residue was treated with Et₃N and then concentrated and washed with water and subjected to FCC (CH₂Cl₂/MeOH, 20:1) to give product as light grey solid 2-chloro-5-(5-((4-methoxybenzo[d]thiazol-2-yl)amino)-1,3,4-oxadiazol-2-yl)benzonitrile 18d (0.15 g, 24%). ¹H NMR (300 MHz, DMSO-d₆): δ 8.45 (s, 1H), 8.44-8.23 (dd, 1H), 7.96-7.93 (d, 1H), 7.46-7.43 (d, 1H), 7.27-7.21 (t, 1H), 7.12-7.09 (d, 1H), 3.95 (s, 3H). LCMS (TOF-ESI) for C₁₇H₁₀ClN₅O₂S, calculated for [M+H]: 384.0314, found for [M+H]⁺: 384.0320.

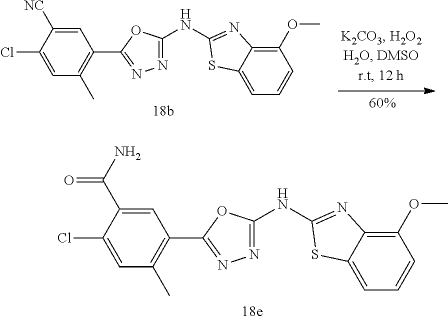 [SMILES: COc1cccc2sc(Nc3nnc(-c4cc(C#N)c(Cl)cc4C)o3)nc12 ; COc1cccc2sc(Nc3nnc(-c4cc(C(N)=O)c(Cl)cc4C)o3)nc12]

### 2-chloro-5-(5-((4-methoxybenzo[d]thiazol-2-yl)amino)-1,3,4-oxadiazol-2-yl)-4-methylbenzamide (18e)

A solution of 2-chloro-5-(5-((4-methoxybenzo[d]thiazol-2-yl)amino)-1,3,4-oxadiazol-2-yl)-4-methylbenzonitrile 18b (40 mg, 0.101 mmol, 1 equiv.) and K₂CO₃ (28 mg, 2 equiv.) in DMSO (2 mL) was added with H₂O₂ (8M in water, 0.1 mL, 5 equiv.), the mixture was stirred at room temperature for 12 h till LC/MS shown the completion of conversion. After completion of reaction, the mixture was filtered and concentrated under high vacuum, the residue was washed with water and dried under high vacuum to provide product 2-chloro-5-(5-((4-methoxybenzo[d]thiazol-2-yl)amino)-1,3,4-oxadiazol-2-yl)-4-methylbenzamide 18e as light yellow solid (25 mg, 60%). ¹H-NMR (600 MHz, d₆-DMSO): δ 8.01 (s, 1H), 7.82 (s, 1H), 7.66 (s, 1H), 7.52 (s, 1H), 7.24-7.22 (d, 1H), 6.90-6.87 (t, 1H), 6.76-6.75 (d, 1H), 3.87 (s, 3H), 2.66 (s, 3H); ¹³C NMR (125 MHz, d₆-DMSO): δ 167.5, 166.7, 155.6, 149.6, 141.9, 138.7, 134.6, 133.9, 132.3, 130.0, 127.0, 123.2, 119.6, 113.1, 107.0, 55.6, 21.3; LCMS (TOF-ESI) for C₁₈H₁₄ClN₅O₃S, Calculated for [M+H]: 416.0579; Found [M+H]⁺ for 416.0574.

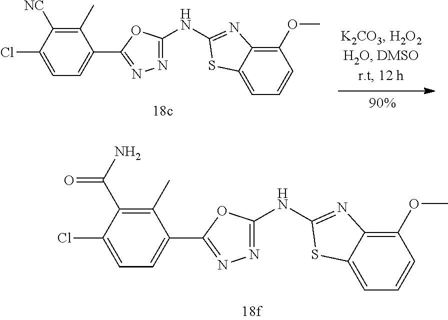 [SMILES: COc1cccc2sc(Nc3nnc(-c4ccc(Cl)c(C#N)c4C)o3)nc12 ; COc1cccc2sc(Nc3nnc(-c4ccc(Cl)c(C(N)=O)c4C)o3)nc12]

### 6-chloro-3-(5-((4-methoxybenzo[d]thiazol-2-yl)amino)-1,3,4-oxadiazol-2-yl)-2-methylbenzamide (18J)

A solution of 6-chloro-3-(5-((4-methoxybenzo[d]thiazol-2-yl)amino)-1,3,4-oxadiazol-2-yl)-2-methylbenzonitrile 18c (40 mg, 0.101 mmol, 1 equiv.) and K₂CO₃ (28 mg, 2 equiv.) in DMSO (2 mL) was added with H₂O₂ (8M in water, 0.1 mL, 5 equiv.), the mixture was stirred at room temperature for 12 h till LC/MS shown the completion of conversion. After completion of reaction, the mixture was filtered and concentrated under high vacuum, the residue was washed with water and dried under high vacuum to provide product 6-chloro-3-(5-((4-methoxybenzo[d]thiazol-2-yl)amino)-1,3,4-oxadiazol-2-yl)-2-methylbenzamide 18f as light yellow solid (37 mg, 90%). ¹H-NMR (600 MHz, d₆-DMSO): δ 8.07 (s, 1H), 7.78 (s, 1H), 7.76-7.75 (d, 1H), 7.47-7.46 (d, 1H), 7.23-7.22 (d, 1H), 6.88-6.86 (t, 1H), 6.75-6.74 (d, 1H), 3.86 (s, 3H), 2.62 (s, 3H); ¹³C NMR (125 MHz, d₆-DMSO): δ 167.9, 166.8, 155.8, 149.6, 141.9, 139.5, 134.1, 134.0, 129.8, 128.1, 126.9, 123.9, 119.5, 113.1, 107.1, 55.6, 18.4; LCMS (TOF-ESI) for C₁₅H₁₄ClN₅O₃S, Calculated for [M+H]: 416.0579; Found [M+H]⁺ for 416.0578.

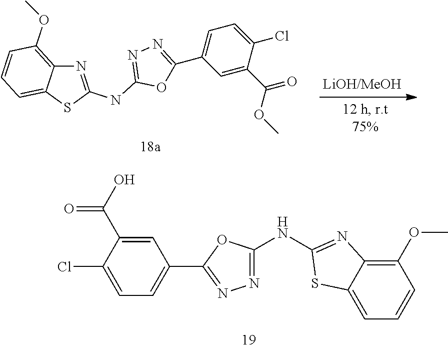 [SMILES: COC(=O)c1cc(-c2nnc(Nc3nc4c(OC)cccc4s3)o2)ccc1Cl ; COc1cccc2sc(Nc3nnc(-c4ccc(Cl)c(C(=O)O)c4)o3)nc12]

### 2-chloro-5-(5-((4-methoxybenzo[d]thiazol-2-yl)amino)-1,3,4-oxadiazol-2-yl)benzoic acid (19)

To a solution of methyl 2-chloro-5-(5-((4-methoxybenzo[d]thiazol-2-yl)amino)-1,3,4-oxadiazol-2-yl)benzoate 18a (40 mg, 0.0961 mmol, 1 equiv.) in MeOH was added with LiOH (20 mg, 5 equiv.), the mixture was stirred at room temperature for 12 hour. Then concentrated to remove most of solvent, TFA (60 mg, 5 equiv.) was added, the mixture was then concentrate to dryness, and diluted with MeOH, the precipitate was collected and dried under high vacuum to provide product 2-chloro-5-(5-((4-methoxybenzo[d]thiazol-2-yl)amino)-1,3,4-oxadiazol-2-yl)benzoic acid 19 as white solid (29 mg, 75%). ¹H-NMR (600 MHz, CDCl₃): δ 13.50 (br. 1H), 8.27 (s, 1H), 8.02-8.01 (d, 1H), 7.73-7.72 (d, 1H), 7.41-7.40 (d, 1H), 7.22-7.19 (t, 1H), 6.07-6.06 (d, 1H), 4.00 (s, 3H), 3.93 (s, 3H), 3.16 (s, 1H); ¹³C NMR (125 MHz, CDCl₃): δ 165.7, 165.2, 158.2, 146.0, 134.1, 132.1, 131.9, 129.1, 127.7, 126.9, 126.6, 123.8, 123.2, 114.5, 108.9, 55.9; LCMS (TOF-ESI) for C₁₅H₁₃N₄O₄ClS, Calculated for [M+H]: 403.0263; Found [M+H]⁺ for 403.0265.

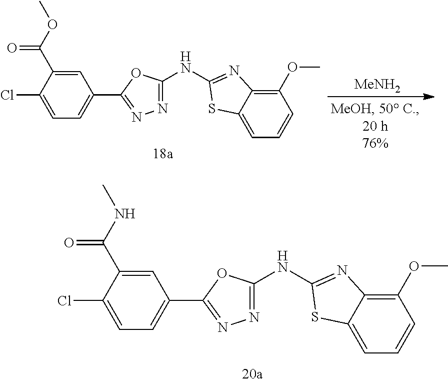 [SMILES: "MeNH2" ; COC(=O)c1cc(-c2nnc(Nc3nc4c(OC)cccc4s3)o2)ccc1Cl ; CNC(=O)c1cc(-c2nnc(Nc3nc4c(OC)cccc4s3)o2)ccc1Cl]

### 2-chloro-5-(5-((4-methoxybenzo[d]thiazol-2-yl)amino)-1,3,4-oxadiazol-2-yl)-N-methylbenzamide (20a)

A solution of methyl 2-chloro-5-(5-((4-methoxybenzo[d]thiazol-2-yl)amino)-1,3,4-oxadiazol-2-yl)benzoate 18a (20 mg, 0.0481 mmol, 1 equiv.) and methylamine (0.2 mL, 40% in water, >20 equiv.) in methanol (2 ml) was stirred for 20 h at 50° C., after completion of reaction, the mixture was concentrated and purified via FCC (5-10% MeOH in CH₂Cl₂) to give product 2-chloro-5-(5-((4-methoxybenzo[d]thiazol-2-yl)amino)-1,3,4-oxadiazol-2-yl)-N-methylbenzamide 20a as off-white solid (16 mg, 76% yield). ¹H-NMR (600 MHz, d₆-DMSO): δ 8.57-8.56 (d, 1H), 7.98-7.96 (dd, 1H), 7.92 (s, 1H), 7.71-7.70 (d, 1H), 7.45-7.43 (d, 1H), 7.24-7.22 (t, 1H), 7.10-7.08 (d, 1H), 3.95 (s, 3H), 2.80 (d, 3H); ¹³C NMR (125 MHz, d₆-DMSO): δ 165.7, 158.4, 137.7, 132.4, 130.8, 127.5, 125.6, 123.8, 123.1, 114.6, 108.9, 56.0, 26.0; LCMS (TOF-ESI) for C₁₈H₁₄N₅O₃SCl, Calculated for [M+H]: 416.0579; Found [M+H]⁺ for 416.0583.

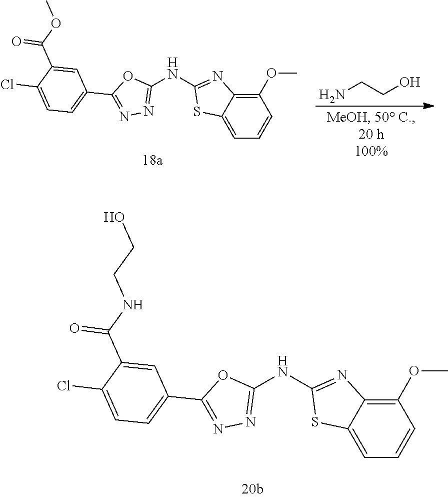

### 2-chloro-N-(2-hydroxyethyl)-5-(5-((4-methoxybenzo[d]thiazol-2-yl)amino)-1,3,4-oxadiazol-2-yl)benzamide (20b)

A solution of methyl 2-chloro-5-(5-((4-methoxybenzo[d]thiazol-2-yl)amino)-1,3,4-oxadiazol-2-yl)benzoate 18a (20 mg, 0.0481 mmol, 1 equiv.) and ethanolamine (0.1 g, 20 equiv.) in methanol (2 ml) was stirred for 20 h at 50° C., after completion of reaction, the mixture was concentrated and purified via FCC (10% MeOH in CH₂Cl₂) to give product 2-chloro-N-(2-hydroxyethyl)-5-(5-((4-methoxybenzo[d]thiazol-2-yl)amino)-1,3,4-oxadiazol-2-yl)benzamide 20b as yellow solid (21 mg, 100% yield). ¹H-NMR (600 MHz, d₆-DMSO): δ 8.62 (s, 1H), 7.87 (s, 1H), 7.85 (s, 1H), 7.63-7.62 (d, 1H), 7.25-7.24 (d, 1H), 6.92-6.90 (t, 1H), 6.78-6.77 (d, 1H), 3.54 (m, 2H), 3.32 (m, 2H); ¹³C NMR (125 MHz, d₆-DMSO): δ 167.2, 166.8, 165.7, 155.4, 149.5, 141.0, 137.6, 133.6, 130.8, 130.5, 126.5, 124.8, 124.2, 119.9, 113.1, 106.9, 59.6, 55.5, 42.0; LCMS (TOF-ESI) for C₁₉H₁₆N₅O₄SCl, Calculated for [M+H]: 446.0685; Found [M+H]⁺ for 446.0682.

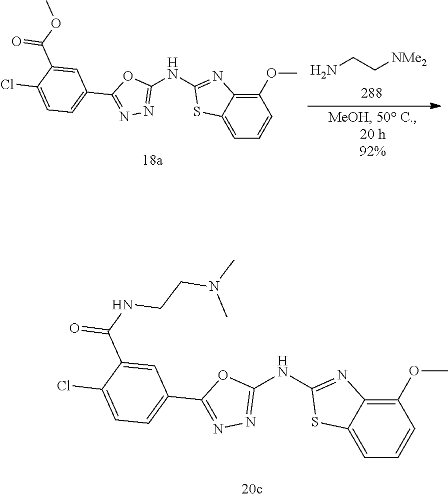

### 2-chloro-N-(2-(dimethylamino)ethyl)-5-(5-((4-methoxybenzo[d]thiazol-2-yl)amino)-1,3,4-oxadiazol-2-yl)benzamide (20c)

A solution of methyl 2-chloro-5-(5-((4-methoxybenzo[d]thiazol-2-yl)amino)-1,3,4-oxadiazol-2-yl)benzoate 18a (20 mg, 0.0481 mmol, 1 equiv.) and amine 288 (0.1 mLg, 20 equiv.) in methanol (2 ml) was stirred for 20 h at 50° C., after completion of reaction, the mixture was concentrated and purified via FCC (5%-10% MeOH in CH₂Cl₂) to give product 2-chloro-N-(2-(dimethylamino)ethyl)-5-(5-((4-methoxybenzo[d]thiazol-2-yl)amino)-1,3,4-oxadiazol-2-yl)benzamide 20c as yellow solid (22 mg, 92% yield). ¹H-NMR (600 MHz, d₆-DMSO): δ 8.72 (s, 1H), 7.94-7.93 (d, 1H), 7.91 (s, 1H), 7.67-7.66 (d, 1H), 7.38-7.37 (d, 1H), 7.14-7.12 (t, 1H), 7.00-6.99 (d, 1H), 3.92 (s, 3H), 3.46 (m, 2H), 2.70 (m, 2H), 2.42 (s, 6H); ¹³C NMR (125 MHz, d₆-DMSO): δ 165.8, 165.6, 157.5, 147.0, 137.5, 131.9, 130.8, 127.3, 125.4, 123.4, 122.7, 114.2, 108.4, 57.3, 55.9, 44.5, 36.6; LCMS (TOF-ESI) for C₂₁H₂₁N₆O₃SCl, Calculated for [M+H]: 473.1157; Found [M+H]⁺ for 473.1166.

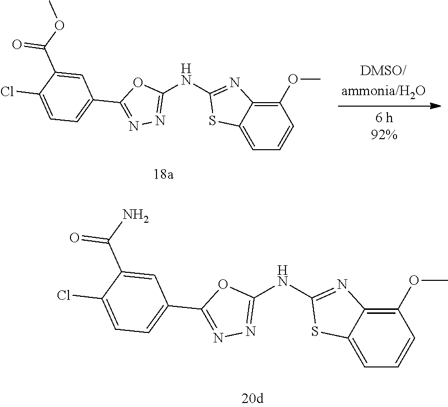 [SMILES: COC(=O)c1cc(-c2nnc(Nc3nc4c(OC)cccc4s3)o2)ccc1Cl ; COc1cccc2sc(Nc3nnc(-c4ccc(Cl)c(C(N)=O)c4)o3)nc12]

### 2-chloro-5-(5-((4-methoxybenzo[d]thiazol-2-yl)amino)-1,3,4-oxadiazol-2-yl)benzamide (20d)

A solution of methyl 2-chloro-5-(5-((4-methoxybenzo[d]thiazol-2-yl)amino)-1,3,4-oxadiazol-2-yl)benzoate 18a (0.2 g, 0.481 mmol, 1 equiv.) and ammonium hydroxide (5 mL, >20 equiv.) in DMSO (10 ml) was stirred for 6 h at room temperature, after completion of reaction, the mixture was diluted with water, the precipitate was collected and washed with water to give product 2-chloro-5-(5-((4-methoxybenzo[d]thiazol-2-yl)amino)-1,3,4-oxadiazol-2-yl)benzamide 20d as pale solid (0.177 g, 92% yield). ¹H-NMR (600 MHz, d₆-DMSO): δ 8.06 (s, 1H), 7.89-7.88 (d, 1H), 7.85 (s, 1H), 7.70 (s, 1H), 7.64-7.62 (d, 1H), 7.27-7.25 (d, 1H), 6.96-6.90 (t, 1H), 6.81-6.79 (d, 1H), 3.87 (s, 3H); LCMS (TOF-ESI) for C₁₇H₁₂ClN₅O₃S, Calculated for [M+H]: 402.0419; Found [M+H]⁺ for 402.0425.

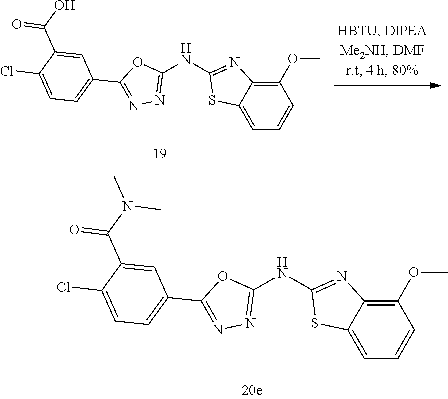 [SMILES: COc1cccc2sc(Nc3nnc(-c4ccc(Cl)c(C(=O)O)c4)o3)nc12 ; COc1cccc2sc(Nc3nnc(-c4ccc(Cl)c(C(=O)N(C)C)c4)o3)nc12]

### 2-chloro-5-(5-((4-methoxybenzo[d]thiazol-2-yl)amino)-1,3,4-oxadiazol-2-yl)-N,N-dimethylbenzamide (20e)

A solution of 2-chloro-5-(5-((4-methoxybenzo[d]thiazol-2-yl)amino)-1,3,4-oxadiazol-2-yl)benzoic acid 19 (20 mg, 0.0497 mmol, 1 equiv.) in DMF (1 mL) was added with HBTU (30 mg, 1.5 equiv.), DIPEA (0.0 4 mL, 3 equiv.) and dimethylamine (2M in THF, 0.04 mL, 1.5 equiv.) at room temperature, the mixture was stirred for 4 h at r.t. The mixture was concentrated and purified via FCC (MeOH/CH₂Cl₂, 2:98) to give solid product 2-chloro-5-(5-((4-methoxybenzo[d]thiazol-2-yl)amino)-1,3,4-oxadiazol-2-yl)-N,N-dimethylbenzamide 20e (17 mg, 80%). ¹H-NMR (600 MHz, CDCl₃): δ 8.03 (s, 1H), 7.98 (m, 2H), 7.54-7.53 (d, 1H), 7.23-7.21 (t, 1H), 7.17-7.16 (d, 1H), 6.90-6.89 (d, 1H), 3.96 (s, 3H), 2.92 (s, 3H), 2.81 (s, 3H); ¹³C NMR (125 MHz, CDCl₃): δ 166.9, 165.1, 162.1, 158.3, 145.6, 136.7, 132.6, 129.9, 127.0, 124.9, 124.4, 123.3, 113.6, 107.7, 55.4, 37.7, 34.3; LCMS (TOF-ESI) for C₁₉H₁₆N₅O₃SCl, Calculated for [M+H]: 430.0735; Found [M+H]⁺ for 430.0745.

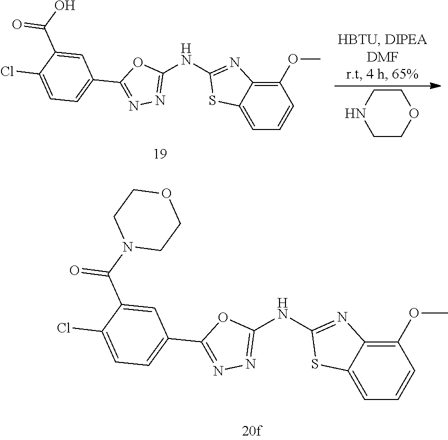 [SMILES: COc1cccc2sc(Nc3nnc(-c4ccc(Cl)c(C(=O)O)c4)o3)nc12 ; C1COCCN1 ; COc1cccc2sc(Nc3nnc(-c4ccc(Cl)c(C(=O)N5CCOCC5)c4)o3)nc12]

### (2-chloro-5-(5-((4-methoxybenzo[d]thiazol-2-yl)amino)-1,3,4-oxadiazol-2-yl)phenyl)(morpholino)methanone (20f)

A solution of 2-chloro-5-(5-((4-methoxybenzo[d]thiazol-2-yl)amino)-1,3,4-oxadiazol-2-yl)benzoic acid 6 (20 mg, 0.0497 mmol, 1 equiv.) in DMF (1 mL) was added with HBTU (30 mg, 1.5 equiv.), DIPEA (0.0 4 mL, 3 equiv.) and morpholine (7 mg, 1.5 equiv.) at room temperature, the mixture was stirred for 4 h at r.t. The mixture was concentrated and purified via FCC (MeOH/CH₂Cl₂, 2:98) to give solid product (2-chloro-5-(5-((4-methoxybenzo[d]thiazol-2-yl)amino)-1,3,4-oxadiazol-2-yl)phenyl)(morpholino)methanone 20f (15 mg, 65%). ¹H-NMR (600 MHz, CDCl₃): δ 8.03 (s, 1H), 8.01-7.99 (d, 1H), 7.98 (s, 1H), 7.56-7.55 (d, 1H), 7.24-7.22 (t, 1H), 7.18-7.17 (d, 1H), 6.91-6.90 (d, 1H), 3.98 (s, 3H), 3.91-3.82 (m, 4H), 3.71-3.64 (m, 2H), 3.34-3.29 (m, 2H); ¹³C NMR (125 MHz, CDCl₃): δ 165.4, 165.2, 162.1, 145.6, 135.7, 132.5, 130.1, 128.5, 127.3, 125.8, 124.9, 124.5, 123.4, 113.6, 107.7, 66.3, 55.4, 46.7, 41.7; LCMS (TOF-ESI) for C₁₉H₁₆N₅O₃SCl, Calculated for [M+H]: 430.0841; Found [M+H]⁺ for 430.0845.

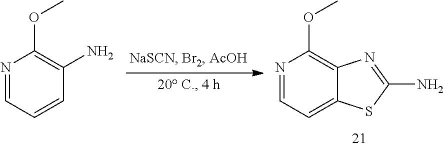

### 4-methoxythiazolo[4,5-c]pyridin-2-amine (21)

To a solution of 2-methoxypyridin-3-amine (10.0 g, 80.6 mmol) in AcOH (100 mL) cooled at about 20° C. was added NaSCN (13.1 g, 161 mmol) in portions, followed by a solution of Bra (4.1 mL, 80.6 mmol) in AcOH (20 mL) dropwise. After addition, the suspension was stirred for 4 h room temperature. The reaction mixture was then poured into ice-water (200 mL) and basified with Na₂CO₃ to pH=9. The resulting mixture was then extracted with EA (3×300 mL). The combined organic layer was washed with sat. aq. Na₂SO₃ (100 mL), brine (2×200 mL), dried over Na₂SO₄ and concentrated. The crude product was purified by column chromatography on silica gel eluted with PE/EA (1/2) to afford desired product 4-methoxythiazolo[4,5-c]pyridin-2-amine 21 (6.2 g, 42%). LC-MS [M+H]⁺ 182. ¹H NMR (300 MHz, DMSO-d₆): δ 7.09 (d, J=7.8 Hz, 1H), 6.93 (d, J=7.8 Hz, 1H), 5.53 (s, 2H), 3.90 (s, 3H). LCMS (TOF-ESI) for C7H₇N₃OS, Calculated for [M+H]: 182.0380; Found [M+H]⁺ for.

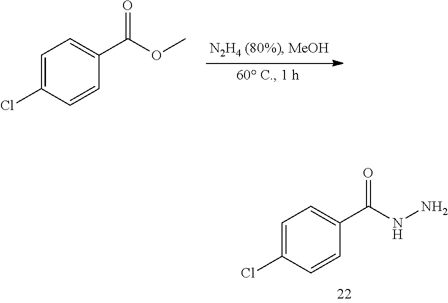

### 4-chlorobenzohydrazide (22)

To a solution of methyl 4-chlorobenzoate (5.0 g, 29.3 mmol) in MeOH (50 mL) was added N₂H₄.H₂O (80%, 50 mL). The resulting solution was heated at 60° C. for 1 h. After concentration, the resulting solid was dissolved in EA (150 mL), washed with brine (2×50 mL), dried over Na₂SO₄ and concentrated to dryness to afford desired product 4-chlorobenzohydrazide 22 (3.2 g, 64%). ¹H NMR (300 MHz, DMSO-d₆): δ 9.83 (s, 1H), 7.85-7.81 (dd, 2 h), 7.54-7.51 (dd, 2H), 4.50 (s, 2H). LCMS (TOF-ESI) for C₇H₇ClN₂O, Calculated for [M+H]: Found [M+H]⁺: 171.

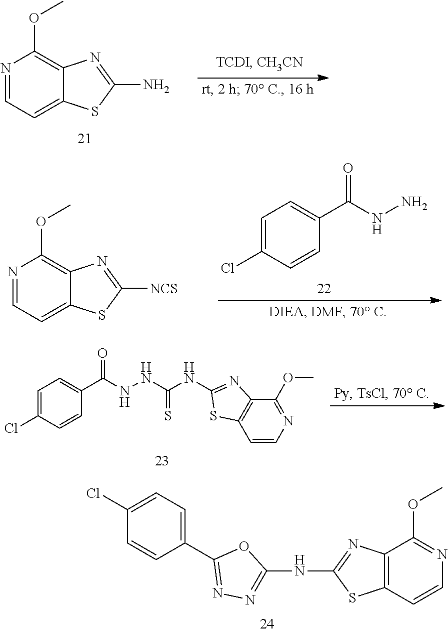

### 5-(4-chlorophenyl)-N-(4-methoxythiazolo[4,5-e]pyridin-2-yl)-1,3,4-oxadiazol-2-amine (24)

To a solution of 4-methoxythiazolo[4,5-c]pyridin-2-amine 21 (100 mg, 0.55 mmol) in CH₃CN (3 mL) was added TCDI (107 mg, 0.60 mmol). After addition, the mixture was stirred for 2 h at room temperature, then heated to 70° C. and stirred for 16 h under N₂. After cooled to room temperature, the solution was concentrated and the residue was dissolved in DMF (3 mL). To the above solution was added 4-chlorobenzohydrazide 22 (102 mg, 0.6 mmol) and DIPEA (155 mg, 1.2 mmol). The resulting solution was heated to 70° C. for 2 h. TLC showed the reaction worked well. After cooled to room temperature, Pyridine (130 mg, 1.65 mmol) and TsCl (157 mg, 0.83 mmol) was added and the solution was heated to 70° C. for 2 h. After cooled to room temperature, the reaction solution was filtered and the filtrate was concentrated and purified by prep-TLC to afford the compound 5-(4-chlorophenyl)-N-(4-methoxythiazolo[4,5-c]pyridin-2-yl)-1,3,4-oxadiazol-2-amine 24. ¹H NMR (300 MHz, DMSO-d₆): δ 10.49 (brs, 1H), 8.52-8.49 (d, 1H), 7.92-7.89 (d, 2H), 7.69-7.66 (d, 2H), 7.43-7.40 (d, 1H), 4.03 (s, 3H). LCMS (TOF-ESI) for C₁₅H₁₀ClN₅O₂S, Calculated for [M+H]: 360.0314; Found [M+H]⁺: 360.0320.

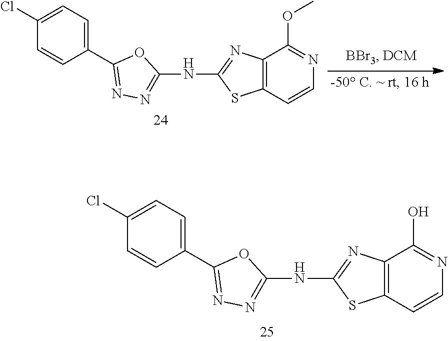 [SMILES: COc1nccc2sc(Nc3nnc(-c4ccc(Cl)cc4)o3)nc12 ; Oc1nccc2sc(Nc3nnc(-c4ccc(Cl)cc4)o3)nc12]

### 2-((5-(4-chlorophenyl)-1,3,4-oxadiazol-2-yl)amino)thiazolo[4,5-c]pyridin-4-ol (25)

To a solution of compound 5-(4-chlorophenyl)-N-(4-methoxythiazolo[4,5-c]pyridin-2-yl)-1,3,4-oxadiazol-2-amine 24 (200 mg, 0.56 mmol) in DCM (20 mL) cooled at −50° C. was added a solution of BBr₃ in DCM (2.2 mL, 1.0 M/DCM, 2.2 mmol) dropwise. After addition, the resulting solution was allowed to warm to room temperature and stirred for 16 h. The reaction was cooled to 0° C. and quenched with H₂O (1 mL). The resulting mixture was concentrated and purified by prep-TLC to afford the compound 2-((5-(4-chlorophenyl)-1,3,4-oxadiazol-2-yl)amino)thiazolo[4,5-c]pyridin-4-ol 25. ¹H NMR (300 MHz, DMSO-d₆): δ 8.26 (d, 1H), 7.92-7.89 (d, 2H), 7.69-7.66 (d, 2H), 7.07 (d, 1H). LCMS (TOF-ESI) for C₁₄H₈ClN₅O₂S, Calculated for [M+H]: 346.0157; Found [M+H]⁺: 346.0163.

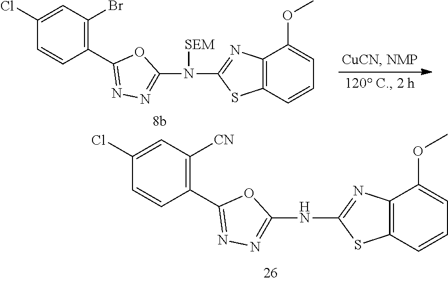 [SMILES: COc1cccc2sc(N(COCC[Si](C)(C)C)c3nnc(-c4ccc(Cl)cc4Br)o3)nc12 ; COc1cccc2sc(Nc3nnc(-c4ccc(Cl)cc4C#N)o3)nc12]

### 5-chloro-2-(5-((4-methoxybenzo[d]thiazol-2-yl)amino)-1,3,4-oxadiazol-2-yl)benzonitrile (26)

To a solution of 5-(2-bromo-4-chlorophenyl)-N-(4-methoxybenzo[d]thiazol-2-yl)-N-((2-(trimethylsilyl)ethoxy)methyl)-1,3,4-oxadiazol-2-amine 8b (140 mg, 0.25 mmol) in NMP (3 mL) was added CuCN (45 mg, 0.5 mmol). After addition, the resulting mixture was stirred for 2 h at 120° C. under Nz. LCMS show desired MS. The reaction mixture was cooled to room temperature, diluted with water, filtered, and dark solid was washed with H₂O, MeOH, DMSO. The solid was further purified by prep-HPLC to afford the compound 5-chloro-2-(5-((4-methoxybenzo[d]thiazol-2-yl)amino)-1,3,4-oxadiazol-2-yl)benzonitrile 26. LCMS (TOF-ESI) for C₁₇H₁₀ClN₅O₂S, Calculated for [M+H]: 384.0314; Found [M+H]⁺: 384.0319.

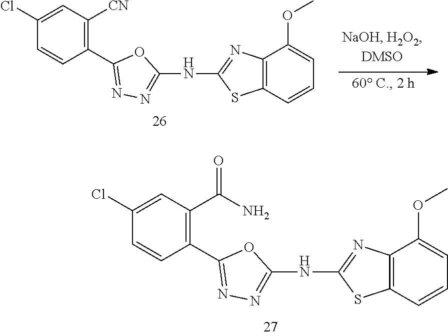 [SMILES: COc1cccc2sc(Nc3nnc(-c4ccc(Cl)cc4C#N)o3)nc12 ; COc1cccc2sc(Nc3nnc(-c4ccc(Cl)cc4C(N)=O)o3)nc12]

### 5-chloro-2-(5-((4-methoxybenzo[d]thiazol-2-yl)amino)-1,3,4-oxadiazol-2-yl)benzamide (27)

To a solution of compound 5-chloro-2-(5-((4-methoxybenzo[d]thiazol-2-yl)amino)-1,3,4-oxadiazol-2-yl)benzonitrile 26 (300 mg, 0.78 mmol) in DMSO (5 mL) was added H₂O₂ (1 mL) dropwise, followed by NaOH (94 mg, 2.34 mmol). After addition, the resulting mixture was stirred for 2 h at 60° C. The reaction mixture was cooled to room temperature and diluted with DMSO (5 mL). After filtration, the filtrate was directly purified by prep-HPLC to the compound 5-chloro-2-(5-((4-methoxybenzo[d]thiazol-2-yl)amino)-1,3,4-oxadiazol-2-yl)benzamide 27 (15 mg). ¹H NMR (300 MHz, DMSO-d₆): δ 8.46-8.44 (d, 1H), 7.95-7.94 (d, 1H), 7.92 (s, 1H), 7.51-7.48 (d, 1H), 7.25-7.19 (t, 1H), 7.00-6.97 (d, 1H), 3.91 (s, 3H). LCMS (TOF-ESI) for C₁₇H₁₂ClN₅O₃S, Calculated for [M+H]: 402.0419; Found [M+H]⁺:402.0419.

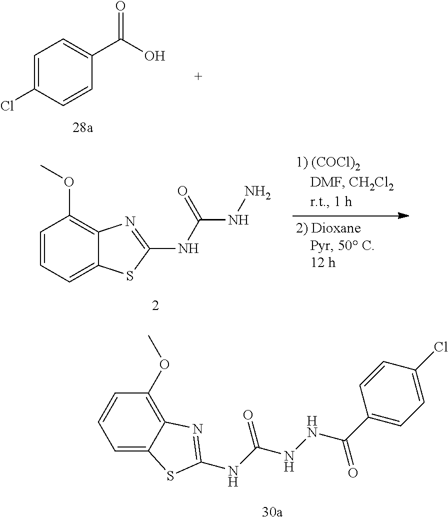 [SMILES: O=C(O)c1ccc(Cl)cc1 ; COc1cccc2sc(NC(=O)NN)nc12 ; COc1cccc2sc(NC(=O)NNC(=O)c3ccc(Cl)cc3)nc12]

### 2-(4-chlorobenzoyl)-N-(4-methoxybenzo[d]thiazol-2-yl)hydrazinecarboxamide (30a)

To a solution of 4-chlorobenzoic acid 28a (1 g, 6.41 mmol, 1 equiv.) and DMF (2 drops) in CH₂Cl₂ (20 mL) was added with oxylyl chloride (COCl)₂ (1 g, 1.2 equiv.) dropwise at room temperature with water bath to maintain the internal temperature at r.t., after completion of addition, the mixture was stirred at room temperature for 1 h. Then evaporated to remove any volatiles and the crude product 4-chlorobenzoyl chloride 29a was used for next step without any purification.

The above obtained 4-chlorobenzoyl chloride 29a was dissolved in dioxane (50 mL) and then hydrazine N-(4-methoxybenzo[d]thiazol-2-yl)hydrazinecarboxamide 268 (1.6 g, 1 equiv.) was added, followed by addition of pyridine (1 g, 2 equiv.). The whole mixture was stirred for 20 h at 50° C., then cooled to room temperature and concentrated to remove any volatiles, the residue was washed with CH₂Cl₂ (20 mL*3) to give product 2-(4-chlorobenzoyl)-N-(4-methoxybenzo[d]thiazol-2-yl)hydrazinecarboxamide 30a as white solid (2 g, 85% over two steps). LCMS (TOF-ESI) for C₁₆H₁₃ClN₄O₃S, Calculated for [M+H]: 377.0467; Found [M+H]⁺ for 377.0485.

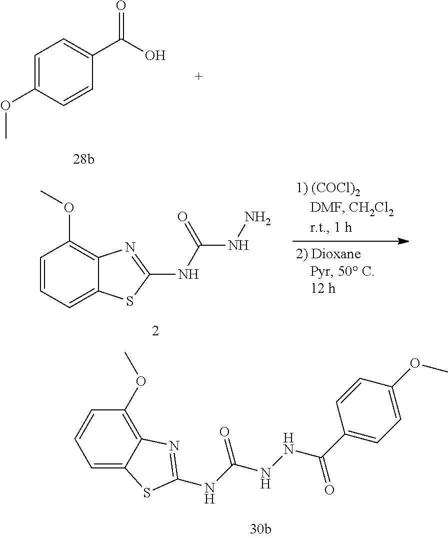 [SMILES: COc1ccc(C(=O)O)cc1 ; COc1cccc2sc(NC(=O)NN)nc12 ; COc1ccc(C(=O)NNC(=O)Nc2nc3c(OC)cccc3s2)cc1]

### N-(4-methoxybenzo[d]thiazol-2-yl)-2-(4-methoxybenzoyl)hydrazinecarboxamide (30b)

To a solution of 4-methoxybenzoic acid 28b (1 g, 6.58 mmol, 1 equiv.) and DMF (2 drops) in CH₂Cl₂ (20 mL) was added with oxylyl chloride (COCl)₂ (1 g, 1.2 equiv.) dropwise at room temperature with water bath to maintain the internal temperature at r.t., after completion of addition, the mixture was stirred at room temperature for 1 h. Then evaporated to remove any volatiles and the crude product 4-methoxybenzoyl chloride 29b was used for next step without any purification.

The above obtained 4-methoxybenzoyl chloride 29b was dissolved in dioxane (50 mL) and then hydrazine N-(4-methoxybenzo[d]thiazol-2-yl)hydrazinecarboxamide 2 (1.6 g, 1 equiv.) was added, followed by addition of pyridine (1 g, 2 equiv.). The whole mixture was stirred for 20 h at 50° C., then cooled to room temperature and concentrated to remove any volatiles, the residue was washed with CH₂Cl₂ (20 mL*3) to give product N-(4-methoxybenzo[d]thiazol-2-yl)-2-(4-methoxybenzoyl)hydrazinecarboxamide 30b as white solid (2 g, 85% over two steps). LCMS (TOF-ESI) for C₁₇H₁₆N₄O₄S, Calculated for [M+H]: 373.0962; Found [M+H]⁺ for 373.0963.

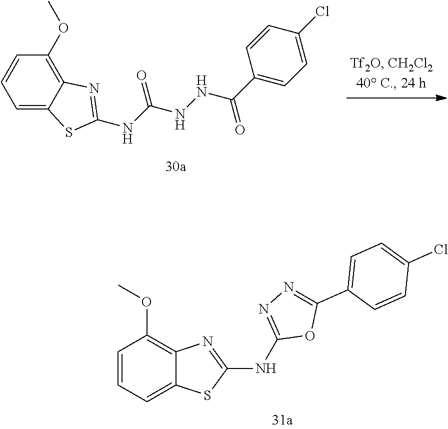 [SMILES: COc1cccc2sc(NC(=O)NNC(=O)c3ccc(Cl)cc3)nc12 ; COc1cccc2sc(Nc3nnc(-c4ccc(Cl)cc4)o3)nc12]

5-(4-chlorophenyl)-N-(4-methoxybenzo[d]thiazol-2-yl)-1,3,4-oxadiazol-2-amine (31a): To a suspension of 2-(4-chlorobenzoyl)-N-(4-methoxybenzo[d]thiazol-2-yl)hydrazinecarboxamide 30a (2 g, 5.32 mmol, 1 equiv.) in CH₂Cl₂ (40 mL) was added with Tf₂O (4.6 g, 3 equiv.) slowly at room temperature. The mixture was then stirred for 24 h at 40° C. After cooling to room temperature, the reaction was filtrated and washed with CH₂Cl₂ (20 mL twice). The cake was dried under high vacuum for 24 h to give product 5-(4-chlorophenyl)-N-(4-methoxybenzo[d]thiazol-2-yl)-1,3,4-oxadiazol-2-amine 31a (1.71 g, 90%). ¹H-NMR (600 MHz, d₆-DMSO): δ 7.94-7.93 (d, 2H), 7.63-7.62 (d, 2H), 7.43-7.41 (d, 1H), 7.23-7.20 (t, 1H), 7.08-7.07 (d, 1H), 3.94 (s, 3H); ¹³C NMR (125 MHz, d₆-DMSO): δ 158.8, 135.6, 129.4, 127.4, 123.8, 123.1, 114.5, 108.9, 56.0; LCMS (TOF-ESI) for C₁₆H₁₁ClN₄O₂S, Calculated for [M+H]: 359.0361; Found [M+H]⁺ for 359.0363.

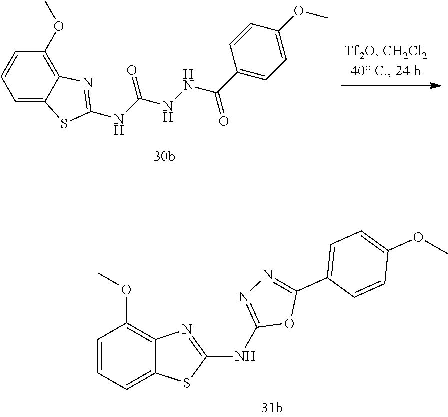 [SMILES: COc1ccc(C(=O)NNC(=O)Nc2nc3c(OC)cccc3s2)cc1 ; COc1ccc(-c2nnc(Nc3nc4c(OC)cccc4s3)o2)cc1]

### N-(4-methoxybenzo[d]thiazol-2-yl)-5-(4-methoxyphenyl)-1,3,4-oxadiazol-2-amine (14)

To a suspension of N-(4-methoxybenzo[d]thiazol-2-yl)-2-(4-methoxybenzoyl)hydrazinecarboxamide 13 (2 g, 7.35 mmol, 1 equiv.) in CH₂Cl₂ (40 mL) was added with Tf₂O (4.6 g, 3 equiv.) slowly at room temperature. The mixture was then stirred for 24 h at 40° C. After cooling to room temperature, the reaction was filtrated and washed with CH₂Cl₂ (20 mL twice). The cake was dried under high vacuum for 24 h to give product N-(4-methoxybenzo[d]thiazol-2-yl)-5-(4-methoxyphenyl)-1,3,4-oxadiazol-2-amine 14 as pale white solid (1.7 g, 90%). NMR (600 MHz, d₆-DMSO): δ 7.89-7.87 (d, 2H), 7.44-7.43 (d, 1H), 7.25-7.22 (t, 1H), 7.13-7.11 (d, 2H), 7.09-7.08 (d, 1H), 3.94 (s, 3H), 3.84 (s, 3H); ¹³C NMR (125 MHz, d₆-DMSO): δ 165.0, 163.7, 161.5, 159.4, 146.1, 141.9, 127.5, 127.3, 127.1, 126.6, 124.0, 116.3, 114.8, 114.6, 108.9, 56.0, 55.4; LCMS (TOF-ESI) for C₁₇H₁₄N₄O₃S, Calculated for [M+H]: 355.0857; Found [M+H]⁺ for 355.0860.

1. A compound of Formula I: 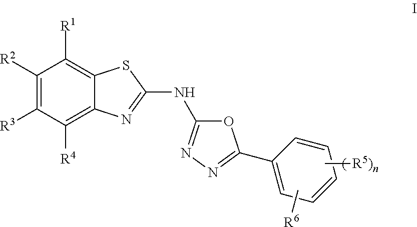
or a pharmaceutically acceptable salt thereof;
R¹, R², R³, and R⁴ are each independently selected from hydrogen, halo, CN, nitro, hydroxy, C₁₋₆ alkyl, C₁₋₆ haloalkyl, C₁₋₆ alkoxy, C₁₋₆ haloalkoxy, amino, C₁₋₆ alkylamino, and di-C₁₋₄-alkylamino;
each R⁵ is independently selected from halo, CN, nitro, hydroxy, C₁₋₆ alkyl, C₁₋₆ haloalkyl, C₁₋₆ alkoxy, C₁₋₆ haloalkoxy, amino, C₁₋₆ alkylamino, and di-C₁₋₄-alkylamino;
n is 0, 1, 2, or 3;
R⁶ is selected from R^{6a}—C(O)— and R^{6b}—C₁₋₄ alkylene-O—;
R^{6a} is selected from C₃₋₇ cycloalkyl, C₂₋₆ heterocycloalkyl, phenyl, and C₁₋₆ heteroaryl, each of which are optionally substituted by 1, 2, 3, or 4 substituents selected from halo, CN, hydroxy, C₁₋₃ alkoxy, amino, C₁₋₃ alkylamino, and di-C₁₋₃-alkylamino;
R^{6b} is selected from —C(O)OR^{a}, —C(O)R^{b}, —C(O)NR^{c}R^{d}, —S(O)₂NR^{c}R^{d}, —OC(O)R^{b}, —NR^{a}C(O)R^{b}, —NR^{a}S(O)₂R^{b}, —NR^{a}C(O)OR^{a}, —OC(O)NR^{c}R^{d}, —NR^{a}C(O)NR^{c}R^{d}, —NR^{a}S(O)₂NR^{c}R^{d}, C₃₋₇ cycloalkyl, C₂₋₆ heterocycloalkyl, phenyl, and C₁₋₆ heteroaryl, wherein said C₃₋₇ cycloalkyl, C₂₋₆ heterocycloalkyl, phenyl, and C₁₋₆ heteroaryl are optionally substituted by 1, 2, 3, or 4 substituents selected from halo, CN, hydroxy, C₁₋₃ alkoxy, amino, C₁₋₃ alkylamino, and di-C₁₋₃-alkylamino;
each R^{a}, R^{c}, and R^{d} is independently selected from hydrogen, C₁₋₄ alkyl, C₃₋₇ cycloalkyl, C₂₋₆ heterocycloalkyl, phenyl, and C₁₋₆ heteroaryl; wherein said C₁₋₄ alkyl, C₃₋₇ cycloalkyl, C₂₋₆ heterocycloalkyl, phenyl, and C₁₋₆ heteroaryl are each optionally substituted by 1, 2, 3, or 4 substituents selected from halo, CN, hydroxy, C₁₋₃ alkoxy, amino, C₁₋₃ alkylamino, and di-C₁₋₃-alkylamino; and
each R^{b} is independently selected from C₁₋₄ alkyl, C₃₋₇ cycloalkyl, C₂₋₆ heterocycloalkyl, phenyl, and C₁₋₆ heteroaryl; each of which is optionally substituted by 1, 2, 3, or 4 substituents independently selected from halo, CN, hydroxy, C₁₋₃ alkoxy, amino, C₁₋₃ alkylamino, and di-C₁₋₃-alkylamino.

2. A compound of claim 1, or a pharmaceutically acceptable salt thereof, wherein
R⁴ is OCH₃ and R¹, R², and R³ are hydrogen.

3. A compound of Formula II: 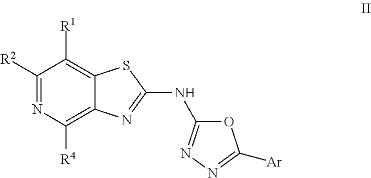
or a pharmaceutically acceptable salt thereof;
Ar is phenyl, which is optionally substituted with 1, 2, 3, 4, or 5 independently selected R^{1a} groups;
R¹, R², and R⁴ are each independently selected from hydrogen, halo, CN, nitro, hydroxy, C₁₋₆ alkyl, C₁₋₆ haloalkyl, C₁₋₆ alkoxy, C₁₋₆ haloalkoxy, amino, C₁₋₆ alkylamino, di-C₁₋₄-alkylamino, carboxy, carbamyl, C₁₋₆ alkylcarbamyl, di(C₁₋₄ alkyl)carbamyl, C₁₋₆ alkylcarbonyl, C₁₋₆ alkoxycarbonyl, C₁₋₆ alkylcarbonyloxy, C₁₋₆ alkylsulfonyl, C₁₋₆ alkylcarbonylamino, C₁₋₆ alkylsulfonylamino, aminosulfonyl, C₁₋₆ alkylaminosulfonyl, di-C₁₋₄ alkylamino sulfonyl, aminosulfonylamino, C₁₋₆ alkylaminosulfonylamino, and di-C₁₋₄ alkylaminosulfonylamino; wherein said C₁₋₆ alkyl, C₁₋₆ haloalkyl, C₁₋₆ alkoxy, C₁₋₆ haloalkoxy, C₁₋₆ alkylamino, di-C₁₋₄-alkylamino, C₁₋₆ alkylcarbamyl, di(C₁₋₄ alkyl)carbamyl, and C₁₋₆ alkylcarbonyl are each optionally substituted with 1, 2, or 3 groups independently selected from halo, CN, hydroxy, C₁₋₃ alkoxy, amino, C₁₋₃ alkylamino, and di-C₁₋₃-alkylamino;
each R^{1a} is independently selected from halo, CN, nitro, hydroxy, C₁₋₆ alkyl, C₁₋₆ haloalkyl, C₂₋₆ alkenyl, C₂₋₆ alkynyl, C₃₋₇ cycloalkyl-C₁₋₃ alkylene, C₂₋₆ heterocycloalkyl-C₁₋₃ alkylene, phenyl-C₁₋₃ alkylene, C₁₋₆ heteroaryl-C₁₋₃ alkylene, C₃₋₇ cycloalkyl, C₂₋₆ heterocycloalkyl, phenyl, C₁₋₆ heteroaryl, C₁₋₆ alkoxy, C₁₋₆ haloalkoxy, amino, C₁₋₆ alkylamino, di-C₁₋₄-alkylamino, carboxy, carbamyl, C₁₋₆ alkylcarbamyl, di(C₁₋₄ alkyl)carbamyl, C₁₋₆ alkylcarbonyl, C₁₋₆ alkoxycarbonyl, C₁₋₆ alkylcarbonyloxy, C₁₋₆ alkylsulfonyl, C₁₋₆ alkylcarbonylamino, C₁₋₆ alkylsulfonylamino, aminosulfonyl, C₁₋₆ alkylaminosulfonyl, di-C₁₋₄ alkylaminosulfonyl, aminosulfonylamino, C₁₋₆ alkylaminosulfonylamino, and di-C₁₋₄ alkylaminosulfonylamino; wherein said C₁₋₆ alkyl, C₁₋₆ haloalkyl, C₂₋₆ alkenyl, C₂₋₆ alkynyl, C₃₋₇ cycloalkyl-C₁₋₃ alkylene, C₂₋₆ heterocycloalkyl-C₁₋₃ alkylene, phenyl-C₁₋₃ alkylene, C₁₋₆ heteroaryl-C₁₋₃ alkylene, C₃₋₇ cycloalkyl, C₂₋₆ heterocycloalkyl, phenyl, C₁₋₆ heteroaryl, C₁₋₆ alkoxy, C₁₋₆ haloalkoxy, C₁₋₆ alkylamino, C₁₋₆ alkylcarbamyl, di(C₁₋₄ alkyl)carbamyl, and C₁₋₆ alkylcarbonyl are each optionally substituted with 1, 2, or 3 groups independently selected R^{1b} groups; and
each R^{1b} group is independently selected from halo, CN, nitro, hydroxy, C₁₋₆ alkyl, C₁₋₆ haloalkyl, C₂₋₆ alkenyl, C₂₋₆ alkynyl, C₃₋₇ cycloalkyl-C₁₋₃ alkylene, C₂₋₆ heterocycloalkyl-C₁₋₃ alkylene, phenyl-C₁₋₃ alkylene, C₁₋₆ heteroaryl-C₁₋₃ alkylene, C₃₋₇ cycloalkyl, C₂₋₆ heterocycloalkyl, phenyl, C₁₋₆ heteroaryl, C₁₋₆ alkoxy, C₁₋₆ haloalkoxy, amino, C₁₋₆ alkylamino, di-C₁₋₄-alkylamino, carboxy, carbamyl, C₁₋₆ alkylcarbamyl, di(C₁₋₄ alkyl)carbamyl, C₁₋₆ alkylcarbonyl, C₁₋₆ alkoxycarbonyl, C₁₋₆ alkylcarbonyloxy, C₁₋₆ alkylsulfonyl, C₁₋₆ alkylcarbonylamino, C₁₋₆ alkylsulfonylamino, aminosulfonyl, C₁₋₆ alkylaminosulfonyl, di-C₁₋₄ alkylaminosulfonyl, aminosulfonylamino, C₁₋₆ alkylaminosulfonylamino, and di-C₁₋₄ alkylaminosulfonylamino.

4. A compound selected from the group consisting of the compounds identified in Examples 3a, 3b, 3c, 8a, 8b, 12a, 12b, 12c, 12d, 15a, 15b, 15c, 15d, 16a, 16b, 16c, 16d, 16e, 18a, 18b, 18c, 18d, 18e, 18f, 20a, 20b, 20c, 20d, 20e, 20f, 26, 27, 31a, and 31b, or a pharmaceutically acceptable salt thereof.

5. A pharmaceutical composition comprising a compound of claim 1, or a pharmaceutically acceptable salt thereof, and a pharmaceutically acceptable carrier.

6. A method of treating a condition selected from the group consisting of heart failure, cardiac hypertrophy, myocarditis, myocardial infarction, ischemia, cardiac arrhythmias, vascular rhexis, cardiac arrhythmia, valvulopathy, diastolic dysfunction, hypertension, cancer, neurodegenerative disorders, viral infection, bacterial infection, liver disease and inflammation in a patient in need thereof, comprising administering to said patient a therapeutically effective amount of a compound of claim 1, or a pharmaceutically acceptable salt thereof.

7. A method according to claim 6, wherein said heart failure is selected from congestive heart failure (CHF), chronic heart failure, and ischemic heart failure.

8. A pharmaceutical composition comprising a compound of claim 2, or a pharmaceutically acceptable salt thereof, and a pharmaceutically acceptable carrier.

9. A pharmaceutical composition comprising a compound of claim 3, or a pharmaceutically acceptable salt thereof, and a pharmaceutically acceptable carrier.

10. A pharmaceutical composition comprising a compound of claim 4, or a pharmaceutically acceptable salt thereof, and a pharmaceutically acceptable carrier.

11. A method of treating a condition selected from the group consisting of heart failure, cardiac hypertrophy, myocarditis, myocardial infarction, ischemia, cardiac arrhythmias, vascular rhexis, cardiac arrhythmia, valvulopathy, diastolic dysfunction, hypertension, cancer, neurodegenerative disorders, viral infection, bacterial infection, liver disease and inflammation in a patient in need thereof, comprising administering to said patient a therapeutically effective amount of a compound of claim 2, or a pharmaceutically acceptable salt thereof.

12. A method according to claim 11 wherein said heart failure is selected from congestive heart failure (CHF), chronic heart failure, and ischemic heart failure.

13. A method of treating a condition selected from the group consisting of heart failure, cardiac hypertrophy, myocarditis, myocardial infarction, ischemia, cardiac arrhythmias, vascular rhexis, cardiac arrhythmia, valvulopathy, diastolic dysfunction, hypertension, cancer, neurodegenerative disorders, viral infection, bacterial infection, liver disease and inflammation in a patient in need thereof, comprising administering to said patient a therapeutically effective amount of a compound of claim 3, or a pharmaceutically acceptable salt thereof.

14. A method according to claim 13 wherein said heart failure is selected from congestive heart failure (CHF), chronic heart failure, and ischemic heart failure.

15. A method of treating a condition selected from the group consisting of heart failure, cardiac hypertrophy, myocarditis, myocardial infarction, ischemia, cardiac arrhythmias, vascular rhexis, cardiac arrhythmia, valvulopathy, diastolic dysfunction, hypertension, cancer, neurodegenerative disorders, viral infection, bacterial infection, liver disease and inflammation in a patient in need thereof, comprising administering to said patient a therapeutically effective amount of a compound of claim 4, or a pharmaceutically acceptable salt thereof.

16. A method according to claim 15 wherein said heart failure is selected from congestive heart failure (CHF), chronic heart failure, and ischemic heart failure.
